(19) United States
(12) Patent Application Publication
(10) Pub. No.: US 2002/0137706 A1
(43) Pub. Date: Sep. 26, 2002

(54) Regulated growth factor delivery for engineered peripheral nerve

(76) Inventors: Gregory R.D. Evans, Tustin, CA (US); Charles W. Patrick JR., Houston, TX (US); Mathias Schmidt, Konstanz (DE); Zhen Fan, Houston, TX (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701, US

(21) Appl. No.: 09/910,681

(22) Filed: Jul. 20, 2001

Related U.S. Application Data
(60) Provisional application No. 60/220,086, filed on Jul. 21, 2000.

(51) Int. Cl. A61K 48/00

(52) U.S. Cl. 514/44; 424/93.21

(57) ABSTRACT

The present invention relates to compositions and methods for the stimulation of nerve cell growth and the regeneration of nerve tissue. Using engineered “helper” cells and nerve growth conduits, in vivo stimulation of nerve cell growth, for example, in damaged or diseased tissues, is achieved.

The application claims priority to U.S. Provisional Application Serial No. 60/220,086, filed on Jul. 21, 2000.

### BACKGROUND OF THE INVENTION

1. Field of the Invention

The present invention relates generally to the fields of molecular biology and neurology. More particularly, it concerns the development of compositions, devices and methods to promote growth of nerve tissue. In specific embodiments, methods are provided to promote repair of injuries to nerve cells.

2. Description of Related Art

Tumor extirpation, traumatic injuries and congenital anomalies may result in injury to or sacrifice of critical nerves. Failure to restore injured nerves can result in the loss of muscle function, impaired sensation and/or painful neuropathies. Functional nerve defects have traditionally been reconstructed by the surgical transfer and sacrifice of healthy normal nerve or muscle from an uninjured location to the injured site. Alternatively, allografts have been attempted in reconstruction despite their requirements for immunosuppression. Despite advances in the outcome of nerve reconstruction, clinicians are still limited by the less than ideal results, and morbidity associated with nerve harvest of autogenous nerve grafts.

Another approach to treatment of nerve injury is use of nerve conduit. Glück initially utilized decalcified bone as a conduit for nerve regeneration. Glück (1880). A variety of other substances have been employed including fascia, vein grafts and fallopian tubes. Wang noted the jugular vein was a better conduit for nerve regeneration than femoral veins which was thought to be due to the increased diameter of the jugular veins. Wang et al. (1992). Silicone tubes have also been used to bridge 10 mm nerve gaps, with compound action potentials detectable by 6 weeks. Lundborg & Hansson (1980). Pseudosynovial sheaths formed around a silicone rod have been employed to fill small nerve gaps. Wang et al. (1992). Unfortunately these conduits were limited to reconstruction of small nerve defects (<10 mm) and functional nerve regeneration following their use could not be assessed. Clinically, nerve defects up to and greater than 200 mm may require repair and restoration of function. Bioabsorable polyglycolic acid (PGA) nerve conduits were compared with the classical sural nerve graft in 16 monkeys 1 year after implantation. The bioabsorbable nerve conduit and the sural nerve graft groups had mean fiber diameters, amplitudes and conduction velocities significantly less than those of normal control ulnar nerves. Dellon and Mackinnon 1988). Further studies utilized poly-L-lactide/poly-L-caprolactone copolymeric nerve guidance channels in the rat sciatic nerve. Nerve conduits were present 2 years after implantation. The mean fiber diameter was smaller in the conduit group compared with controls. den Dunner et al. (1996).

Attempts to bridge larger nerve deficits a variety of substances have been used to fill these conduits. Williams noted that silicone tubes filled with dialyzed plasma resulted in a three to five fold increase in functional restitution detected at 8 weeks compared with chambers prefilled with phosphate buffered saline (PBS). Williams et al. (1987). For a 20-25 mm sciatic nerve gap in rats, Madison filled silicone tubes with collagen or laminin and compared them with empty control tubes. All of the tubes with additives demonstrated nerve regeneration extending up to 4-6 mm. Madison et al. (1988). Archibald compared autografting and entubulation repair with collagen based nerve guide conduits across 4 mm gaps in both rats and monkeys. Archibald et al. (1991). They noted that recovery of physiological response from target muscle and sensory nerve entubulation repair with a collagen-based nerve guide conduits was as effective as a standard nerve autograft over short nerve defects. Archibald et al. (1991). Seckel used hyaluronic acid, a compound associated with decreased scarring and improved fibrin matrix formation, in an injectable polyethylene nerve guide in the rat sciatic nerve. Seckel et al. (1984). Better conduction velocity, higher axon counts, and a trend toward earlier myelination was demonstrated with hyaluronic acid compared with saline. Seckel et al. (1984). Contrary to these study results, Valentini demonstrated that semipermeable guidance channels filled with collagen or laminin displayed fewer myelinated axons than saline. Valentini et al. (1987).

Previous attempts to used poly(L-lactic-co-glycolic acid) (PLGA) and poly(L-lactic acid) (PLLA) biodegradable polymer conduits were used to assess applicability for in vivo use. Evans et al. (1998); Evans et al. (1999); Widmer et al. (1998). Schwann cells (SC's) with peripheral nerves were used as active support cells. However, the SC's implanted showed varied results. In additions, adult SC's are difficult to culture into clinical quantities ex vivo in a reasonable time frame. Thus, there remain limitations on the ability to use nerve scaffolds for nerve replacement in vivo.

### SUMMARY OF THE INVENTION

Thus, in accordance with the present invention, there is provided a method for regenerating nerve tissue in vivo comprising (a) providing a device comprising a biodegradable conduit comprising at least two openings and a passage connecting said openings and fibroblast cells transformed with an expression cassette comprising a promoter, active in eukaryotic cells, that directs the expression of a polynucleotide encoding nerve growth factor (NGF), wherein said fibroblast cells are disposed within said passage, and (b) implanting said device in a subject such that each of said openings are adjacent to nerve tissues, whereby said nerve tissues are stimulated to regenerate into said passage by NGF produced by said fibroblast cells. The promoter may be CMV IE, SV40, HSV tk, RSV LTR, EF-1α or ubiquitin. The expression construct may further comprise one or more of a polyadenylation signal, a selectable marker or a screenable marker.

Fibroblast cells may be dermal fibroblast cells. The biodegradable conduit may be comprised of PLGA or PLLA. The NGF expression may be inducible, for example, by Muristerone A, GS-E, or tetracycline. The administration of these inducers may be intravenous, intrathecal, intracavitary and by catheter, and may be for 24 hours, 48 hours, four days, seven days, ten days or longer. The fibroblast cells may further comprise a cell kill gene that renders said fibroblast cells susceptible to killing following administration of a substance. In one embodiment, the kill gene encodes an enzyme and said substance is a prodrug. The kill gene may comprise a promoter selected from the group consisting of CMV IE, SV40, HSV tk, RSV LTR, EF-1α and ubiquitin. In a specific embodiment, the the cell kill gene is thyinidine kinase. In another embodiment, the cell kill gene is a toxin and said substance is an activator of the transcription of said cell kill gene.

In another embodiment, there is provided an implantable device comprising (a) a biodegradable conduit comprising at least two openings and a passage connecting said openings; and (b) fibroblast cells transformed with an expression cassette comprising a promoter, active in eukaryotic cells, that directs the expression of a polynucleotide encoding nerve growth factor (NGF), wherein said fibroblast cells are disposed within said passage.

In yet another embodiment, there is provide a kit comprising an implantable device comprising (a) a biodegradable conduit comprising at least two openings and a passage connecting said openings; and (b) fibroblast cells transformed with an expression cassette comprising a promoter, active in eukaryotic cells, that directs the expression of a polynucleotide encoding nerve growth factor (NGF), wherein said fibroblast cells are disposed within said passage. The kit may further comprise an inducer for the promoter in a suitable container. The kit also may comprise a substance that selectively kills said cell, and said substance in a suitable container.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Transient transfection efficiency of β-Gal transfected DFBs. Indirect methods included FuGENE6, Transfectam, DOTAP and ESCORT. The direct method utilized a gene gun operated at 100 psi (Gene Gun I) and 200 psi (Gene Gun II). Data represent mean±SEM performed in duplicate.

FIG. 2—NGF release from DFBs transfected with an expression vector encoding rat β-NGF (pSec tag NGF), vector alone (pSec tag), and nothing (control). Data represent mean±performed in quadruplicate (n=4). H denotes a statistically significant difference of NGF released from NGF transfected DFB's compared to control vector transfected DFB's at all time points (p≦0.002). # denotes a statistically significant difference in NGF released from NGF transfeced DFB's at 72 hrs compared to 24 hrs (p≦0.002) or 48 hrs (p≦0.01). § denotes a statistically significant difference in NGF released from control DFB's at 24 hrs compared to 48 hrs (p≦0.001) or 72 hrs (p≦0.001). A denotes a statistically significant difference in HGF released from vector transfected DFB's at 24 hrs compared to 48 hrs (p≦0.01) or 72 hrs (p≦0.01). π denotes a statistically significant difference in NGF released from control and vector transfected DFB's at all time points (p≦0.002).

FIG. 3—Normalized NGF release from DFB's transfected with an expression vector encoding rat β-NGF (pSec tag NGF), vector alone (pSec tag), and nothing (control). Data represent mean±SEM performed in quadruplicate (n=4). H denotes a statistically significant difference of NGF released from NGF transfected DFB's compared to control vector transfected DFB's at all time points (p≦0.001). # denotes a statistically significant difference in NGF released from NGF transfected DFB's at 48 hrs compared to 24 hrs (p≦0.001) or 72 hrs (p≦0.01). § denotes a statistically significant difference in NGF released from control DFB's at 72 hrs compared to 24 hrs (p≦0.001) or 48 hrs (p≦0.009).

FIG. 4—Release rates of DFB's transfected with an expression vector encoding rat β-NGF (pSec tag NGF), vector alone (pSec tag), and nothing (control). Data represent mean±SEM performed in quadruplicate (n=4). The release rate curve of NGF transfected DFB's is significantly different than curves from control and vector transfected DFB's (p≦0.001).

FIG. 5 Normalized in vitro NGF release from transfected hDFBs in the presence (MurA (+)) or absence (MurA (−), control) of 3 pM Muristerone A. Data represent mean+SEM performed in quadruplicate (n=4). H denotes a statistically significant difference of NGF released from MurA (+) hDFBS compared to control MurA (−) hDFBs at all time points (p≦0.001). † denotes a statistically significant difference in NGF released from MurA (−) between all three time points (p<0.001). ‡ denotes a statistically significant difference in NGF released from MurA (+) hDFBs at 72 h compared to 24 hrs or 48 hrs (p≦0.001).

FIG. 6—In vitro release rates of transfected hDFBs in the presence (MurA (+)) or absence (MurA (−), control) of 3 μM Muristerone A. Data represent mean+SEM performed in quadruplicate (n=4). The release rate curve of MurA (+) hDFBs is significantly different than the curve from MurA(−) hDFBs (p≦0.001).

FIG. 7—Bioactivity assay of released NGF. PC-12 cells were incubated with media (control) or media supernatants from transfected hDFBs in the presence of MurA (i.e., media with released NGF). Data are reported mean±SEM. 1-1 denotes a statistically significant difference in PC-12 differentiation (p≦0.002).

FIG. 8—In vivo NGF release assayed from collection chambers filled with transfected hDFBs with Muristerone A (TFB/MurA (+)), transfected hDFBs without Muristerone A (TFB/MurA (−)), untransfected hDFBs (NTFB), or phosphate buffered saline (PBS). Collection chambers were implanted for 1 and 2 days. Data represent mean±SEM. H and † denote a statistically significant difference of TFB/MurA (+) compared to all other groups at day 1 and 2, respectively.

### DESCRIPTION OF ILLUSTRATIVE EMBODIMENTS

Peripheral nerve injuries can result from mechanical, thermal, chemical, congenital or pathological etiologies. Failure to restore damaged nerves can lead to loss of muscle function, impaired sensation and painful neuropathies. Current surgical strategies for repair of critical nerves involves the transfer of normal donor nerve from an uninjured body location. However, these “gold standard” method for tissue restoration are limited by tissue availability, risk of disease spread, secondary deformities and potential differences in tissue structure and size. One possible alternative to autogenous tissue replacement is the development of engineered constructs to replace those elements necessary for axonal proliferation and include a scaffold, support cells, induction factors and extracellular matrices (ECM's). Despite advances and contributions in the filed of tissue engineering, results to date with nerve conduits have failed to equal nerve regeneration realized with autogenous grafts for large distances. In our current attempts to focus on engineered constructs necessary for neural tissue replacement, the present inventors have identified a novel approach to deliver nerve-specific growth factors.

The present invention employs molecular strategies to deliver growth factors to injured nerve tissue. In particular, this is done in the context of a biodegradable polymer conduit, using dermal fibroblasts to deliver nerve growth factor (NGF). NGF promotes interactions between axons and Schwann cells (SC), thereby enhancing peripheral nerve regeneration.

I. NERVE CELL BIOLOGY

The nervous system facilitates communication between different parts of the body. It also controls responses to stimuli and controls complex behavior involving numerous aspects. The nervous system also is capable of learning—as it processes information, it undergoes changes that permit altered futures patterns of both action and reaction.

The basic element of the nervous system is the neuron. Neurons are elongated cells that receive, conduct and transmit signals. Their length, ranging up to a meter, is an important attribute in moving signals from distal body parts to the brain and spinal column, and back again. Neurons have cell body, containing the nucleus, and a number of long, thin processes radiating outward from it. Typically, a single axon is present and is responsible for sending signals away from the neuron. The axon may divide at its terminus. Multiple dendrites, which are shorter processes, extend from the cell body and serve to receive signals. Dendrites from a single cell can number in the thousands.

Neuronal signals are transmitted from one cell to another at specialized sites of connection called synapses. In a presynaptic (upstream) cell, an electrical impulse results in the release of a neurotransmitter which crosses the synaptic junction and provoke an electrical change in the postsynaptic (downstream) cell. A neuron also communicates within itself through vesicular (fast) and microtubule (slow) transport.

Surrounding all neuronal tissues are supporting cells called glial cells. Glial cells surround the neurons and even fill spaces between the neurons. The best understood are Schwann cells in vertebrate peripheral nerves, and oligodendrocytes in vertebrate central nervous systems. These cells provide electrical insulation for the neurons and comprise a structure known as the myelin sheath. Three other types of glial cells exist: microglia (functionally related to macrophages); ependymal cells (lining for inside of brain and spinal cord); astrocytes (development).

When nervous tissue is damaged, connections between the neurons cells may be disrupted. In addition, glial cell structures can be damaged. However, unlike glial cells, neurons have no capacity to divide, and thus the only real hope for repair of neuronal tissue is to stimulate growth in such a way that connections between neurons can be reestablished. As discussed below, nerve cell growth is highly dependent upon a number of factors, including nerve promoting growth factors and physical supports upon which nerve regrowth can be made.

II. NERVE PROMOTING GROWTH FACTORS

In accordance with the present invention, any nerve promoting growth factors can be used to stimulate nerve cell growth. In particular, applicants will utilize Nerve Growth Factor (NGF). However, other factors such as Fibroblast Growth Factor (FGF), Brain-Derived Neurotrophic Factor (BDNF), GDNF, VEGF, neurotrophin 3, neurotrphin 4-5 and a variety of other neural receptors including Trks receptors may be used.

III. HELPER CELLS

As discussed above, an important feature of the present invention is the use of host cells which provide growth factors to neighboring nerve cells. Generally, such cells would include those with the ability to release growth factors discussed above, to provide signal transduction like that seen in normal nerve tissues, and not present any growth restricting attributes.

A. Host Cells

As used herein, the terms “cell,” “cell line,” and “cell culture” may be used interchangeably. All of these terms also include their progeny, which are any and all subsequent generations. It is understood that all progeny may not be identical due to deliberate or inadvertent mutations. In the context of expressing a heterologous nucleic acid sequence, “host cell” refers to a eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be selected depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result.

Examples of eukaryotic host cells for use in accordance with the present invention include fibroblast cells, stem cells, fat cells, Schwann cells, astrocytes, endothelial cells and ex vivo propagated nerve cells. These and other cells may be encapsulated in various biocompatible matrices to suppress potential immunogenicity.

B. Expression Constructs

1. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 1 lists several elements/promoters that may be employed, in the context of the present invention, to regulate the expression of a gene. This list is not intended to be exhaustive of all the possible elements involved in the promotion of expression but, merely, to be exemplary thereof. Table 2 provides examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin Heavy Chain | Banerji et al., 1983; Gilles et al., 1983; |
| | Grosschedl et al., 1985; Atchinson et |
| | al., 1986, 1987; Imler et al., 1987; |
| | Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; |
| | Redondo et al.; 1990 |
| HLA DQ a and/or DQ β | Sullivan et al., 1987 |
| β-Interferon | Goodbourn et al., 1986; Fujita et al., |
| | 1987; Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; |
| | Johnson et al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., |
| | 1989, 1990 |
| α-Fetoprotein | Godbout et al., 1988; Campere et al., |
| | 1989 |
| t-Globin | Bodine et al., 1987; Perez-Stable et al., |
| | 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth | Pech et al., 1989 |
| Factor (PDGF) | |
| Duchenne Muscular Dystrophy | Klamut et al., 1990 |
| SV40 | Banerji et al., 1981; Moreau et al., |
| | 1981; Sleigh et al., 1985; Firak et al., |
| | 1986; Herr et al., 1986; Imbra et al., |
| | 1986; Kadesch et al., 1986; Wang et al., |
| | 1986; Ondek et al., 1987; Kuhl et al., |
| | 1987; Schaffner et al., 1988 |
| Polyoma | Swartzendruber et al., 1975; Vasseur |
| | et al., 1980; Katinka et al., 1980, 1981; |
| | Tyndell et al., 1981; Dandolo et al., |
| | 1983; de Villiers et al., 1984; Hen et al., |
| | 1986; Satake et al., 1988; Campbell |
| | and/or Villarreal, 1988 |
| Retroviruses | Kriegler et al., 1982, 1983; Levinson |
| | et al., 1982; Kriegler et al., 1983, |
| | 1984a, b, 1988; Bosze et al., 1986; |
| | Miksicek et al., 1986; Celander et al., |
| | 1987; Thiesen et al., 1988; Celander et |
| | al., 1988; Chol et al., 1988; Reisman |
| | et al., 1989 |
| Papilloma Virus | Campo et al., 1983; Lusky et al., 1983; |
| | Spandidos and/or Wilkie, 1983; |
| | Spalholz et al., 1985; Lusky et al., |
| | 1986; Cripe et al., 1987; Gloss et al., |
| | 1987; Hirochika et al., 1987; Stephens |
| | et al., 1987; Glue et al., 1988 |
| Hepatitis B Virus | Bulla et al., 1986; Jameel et al., 1986; |
| | Shaul et al., 1987; Spandau et al., 1988; |
| | Vannice et al., 1988 |
| Human Immunodeficiency Virus | Muesing et al., 1987; Hauber et al., |
| | 1988; Jakobovits et al., 1988; Feng et |
| | al., 1988; Takebe et al., 1988; Rosen et |
| | al., 1988; Berkhout et al., 1989; Laspia |
| | et al., 1989; Sharp et al., 1989; |
| | Braddock et al., 1989 |
| Cytomegalovirus (CMV) | Weber et al., 1984; Boshart et al., 1985; |
| | Foecking et al., 1986 |
| Gibbon Ape Leukemia Virus | Holbrook et al., 1987; Quinn et al., |
| | 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; Lee et |
| mammmary tumor | | al., 1981; Majors et al., |
| virus) | | 1983; Chandler et al., 1983; |
| | | Ponta et al., 1985; Sakai |
| | | et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1983, herein incorporated by reference.)

5. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

6. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

7. Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Method of Cellular Transformation

Suitable methods for transformation for use with the current invention are believed to include virtually any method by which DNA can be introduced into a cell. Generally, such methods can be grouped as either viral or non-viral. Through the application of techniques such as these, cells may be stably transformed. The methods will be discussed below.

In particular embodiments, the present inventors use the FuGENE™ 6 non-liposomal transfection methods (Boehringer Mannhein, Germany).

1. Viral Transformation

Adenoviral Infection. One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Retroviral Infection. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

AAV Infection. Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

Other Viral Vectors. Other viral vectors may be employed as constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990). Alternatively, Alphavirus vectors and replicons may be employed (Leitner et al., 2000; Caley et al., 1999).

A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been sugested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Non-Viral Delivery

In addition to viral delivery of the self gene, the following are additional methods of recombinant gene delivery to a given host cell and are thus considered in the present invention.

Electroporation. In certain preferred embodiments of the present invention, the gene construct is introduced into the dendritic cells via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

It is contemplated that electroporation conditions for dendritic cells from different sources may be optimized. One may particularly wish to optimize such parameters as the voltage, the capacitance, the time and the electroporation media composition. The execution of other routine adjustments will be known to those of skill in the art.

Particle Bombardment. Another embodiment of the invention for transferring a naked DNA construct into cells involves particle bombardment. This method depends on the ability to accelerate DNA-coated microprojectiles to a high velocity allowing them to pierce cell membranes and enter cells without killing them (Klein et al., 1987). The microprojectiles used have consisted of biologically inert substances such as tungsten, platinum or gold beads.

It is contemplated that in some instances DNA precipitation onto metal particles would not be necessary for DNA delivery to a recipient cell using particle bombardment. It is contemplated that particles may contain DNA rather than be coated with DNA. Hence it is proposed that DNA-coated particles may increase the level of DNA delivery via particle bombardment but are not, in and of themselves, necessary.

Several devices for accelerating small particles have been developed. One such device relies on a high voltage discharge to generate an electrical current, which in turn provides the motive force (Yang et al., 1990). Another method involves the use of a Biolistic Particle Delivery System, which can be used to propel particles coated with DNA through a screen, such as stainless steel or Nytex screen, onto a filter surface covered with cells in suspension. The screen disperses the particles so that they are not delivered to the recipient cells in large aggregates. It is believed that a screen intervening between the projectile apparatus and the cells to be bombarded reduces the size of projectile aggregates and may contribute to a higher frequency of transformation by reducing the damage inflicted on the recipient cells by projectiles that are too large.

For the bombardment, cells in suspension are preferably concentrated on filters, or alternatively on solid culture medium. The cells to be bombarded are positioned at an appropriate distance below the macroprojectile stopping plate. If desired, one or more screens are also positioned between the acceleration device and the cells to be bombarded.

In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity or either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of primordial germ cells.

Accordingly, it is contemplated that one may wish to adjust various of the bombardment parameters in small scale studies to fully optimize the conditions. One may particularly wish to adjust physical parameters such as gap distance, flight distance, tissue distance and helium pressure. One may also optimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art.

Calcium Phosphate Co-Precipitation or DEAE-Dextran Treatment. In other embodiments of the present invention, the transgenic construct is introduced to the cells using calcium phosphate co-precipitation. Mouse primordial germ cells have been transfected with the SV40 large T antigen, with excellent results (Watanabe et al., 1997). Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

Direct Microinjection or Sonication Loading. Further embodiments of the present invention include the introduction of the gene construct by direct microinjection or sonication loading. Direct microinjection has been used to introduce nucleic acid constructs into Xenopus oocytes (Harland and Weintraub, 1985), and LTK fibroblasts have been transfected with the thymidine kinase gene by sonication loading (Fechheimer et al., 1987).

Liposome Mediated Transformation. In a further embodiment of the invention, the gene construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL) or DOTAP-Cholesterol formulations.

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

Other Non-Viral Methods. Other possible delivery methods include PEG-mediated transformation of protoplasts (Omirulleh et al., 1993), by desiccation/inhibition-mediated DNA uptake (Potrykus et al, 1985), and by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety).

IV. BIODEGRADABLE CONDUITS

The biodegradable conduits of the present invention, also referred to herein as “scaffolds,” have at least two openings and a passageway connecting the openings. The exterior of the present conduits may possess any shape suitable for the application. For example, the exterior of the present conduits may be tubular in shape. The wall of the passageway of the present conduits (i.e., the interior) also may possess any shape suitable for the application. For example, cross sections taken at different locations along the length of the present conduits may have differing areas, revealing an irregularly-shaped interior. This may be equally true of the exterior of the present conduits.

The present biodegradable conduits may be made from a variety of suitable materials. For example, poly-L-lactic acid (PLLA) polymers are attractive candidates for fabricating the present conduits because they are biocompatible, able to hold suture, and biodegrade into naturally metabolized products. Thomas et al. (1995). Three-dimensional porous PLLA foam conduits may be fashioned using a solvent casting and particulate (salt) leaching technique known in the art. These foam conduits degrade by simple, nonenzymatic hydrolysis at a rate related to the crystallinity and copolymer ratio. Evans et al. (1999); Widmer et al. (1998); Mikos et al. (1994); Thomas et al. (1995). PLLA constructs with specific porosity, surface/volume ratio, pore size, and crystallinity may be produced to meet specific engineering requirements.

In contrast to previous studies with variable conduits, the present PLLA scaffolds 1) are fully degradable and are replaced by myelinated axons with degradation controlled and regulated to variable times, 2) are porous to allow vascularization, 3) create a conduit that can be varied in length and luminal diameter while maintaining structural integrity and flexibility, and 4) are fabricated by a unique process that reproduces consistent geometric conformity and added stability. Widmer et al. (1998); Mikos et al. (1994). Moreover, the present PLLA conduits afford surgeons the ability to assess functional outcomes of peripheral nerve regeneration.

Other materials that may be used for the present biodegradable conduits are described in U.S. Pat. No. 5,939,323 to Valentini et al. (1999), which is expressly incorporated by reference herein, and are detailed below. Conduits from the following materials are suitable for a variety of clinical uses, and include bone, cartilage, and soft tissue repair. Conduits formed from the following materials also may be used to promote tissue culture of committed cells and/or differentiation of precursor cells. Thus, the present biodegradable conduits made from the following materials may be used in virtually all instances when it is desirable to provide a substrate for the growth of cells onto or into a tissue replaceable matrix.

U.S. Pat. No. 5,939,323 describes three-dimensional biodegradable scaffolds of hyaluronic acid derivatives for tissue reconstruction and repair. These scaffolds have interconnected pores that permit cells to grow into the scaffold. The cells may completely penetrate the scaffold thereby eventually replacing the scaffold with tissue. The scaffold may be fabricated to be virtually any shape, size or thickness, and may be produced to various porosities and pore sizes, depending upon the application. The scaffolds are degradable, so that eventually they may be completely replaced by tissue. The scaffolds degrade slowly in concert with new tissue formation. Such scaffolds promote host cells to migrate, adhere, proliferate and synthesize new tissue inside the pores, thereby accelerating, for example, wound healing. Void volumes for the present conduits made from the following materials may range from 40-90%. Pore sizes may range from 10-1000 micrometers.

The present conduits use hyaluronic acid derivatives that are water-insoluble, but are soluble in a first solvent. The water-insoluble hyaluronic acid is dissolved in the first solvent, together with a pore forming agent that is insoluble in the first solvent. This mixture is then contacted with a second solvent in which the hyaluronic acid derivative is insoluble, but in which the pore forming agent is soluble. In this manner, the first solvent is replaced/extracted by the second solvent in which the hyaluronic acid is insoluble, bringing the hyaluronic acid derivative out of solution and forming a conduit. Likewise, the pore forming agent is soluble in the second solvent and is extracted/dissolved, thereby leaving a porous scaffold of the water-insoluble hyaluronic acid derivative.

The water-insoluble hyaluronic acid derivatives are known to those skilled in the art and described in numerous publications. For example, because hyaluronic acid is a polycarboxylic acid, its water-insoluble esters may be prepared using standard methods for the esterification of carboxylic acids, such as the treatment of free hyaluronic acid with the desired water-insoluble moieties in the presence of appropriate catalysts. Alternatively, the esters may be prepared by treating a quaternary ammonium salt of hyaluronic acid with an esterifying agent in a suitable aprotic solvent. Details of this latter method have been described in European Patent Application No. EP 216 453, Apr. 1, 1987, the disclosure of which is incorporated herein by reference. Esterification of hyaluronic acid with suitable water-insoluble moieties may also be achieved by the use of linking groups interposed between the hyaluronic acid and the water-insoluble moiety.

Likewise, hyaluronic acid may be derivatized via amide bonds, as will be clear to those skilled in the art. Such hyaluronic acid derivatives are described in the following PCT publications, each of the disclosures of which are incorporated herein by reference. WO95/24429 discloses highly reactive esters of carboxy polysaccharides, including hyaluronic acid. PCT Patent applications WO95/24497 and WO95/04132 disclose methods for preparing high molecular weight hyaluronic acid derivatives.

Hyaluronic acid is a linear polysaccharide. Many of its biological effects are a consequence of its ability to bind water, in that up to 500 ml of water may associate with 1 gram of hyaluronic acid. Esterification of hyaluronic acid with uncharged organic moieties reduces the aqueous solubility. Complete esterification with organic alcohols such as benzyl renders the hyaluronic acid derivatives virtually insoluble in water, these compounds then being soluble only in certain aprotic solvents.

When films of hyaluronic acid are made, the films essentially are gels which hydrate and expand in the presence of water (hydrogels). By esterifying the hyaluronic acid and making it insoluble in water, the present conduits then are possible. The scaffolds are not hydrated in the presence of water and maintain their overall structure, permitting cell ingrowth. Thus, the hyaluronic acid derivatives that are useful for present purposes are those sufficiently derivatized such that the hyaluronic acid derivative will not form a hydrogel. Those of ordinary skill in the art can easily test whether sufficient derivitization with an uncharged moiety has occurred so as prevent the formation of a hydrogel. One hyaluronic derivative is 100% esterified hyaluronic acid-benzyl covalent conjugates, sold under the trade name HYAFF by Fidia Advanced Biopolymers, Abano Terme, Italy.

Solvents for the water-insoluble derivatized hyaluronic acid molecules include dimethylsulfoxide (DMSO), N-methyl-pyrrolidone (NMP), 1, 1, 1, 3, 3, 3-hexafluoro-2-propanol (HFIP) and dimethylacetamide (DMAC). Other appropriate solvents will be known to those of ordinary skill in the art.

Non-solvents for the derivatized hyaluronic acid that may be used include water, ethanol, isopropanol, glycerol, ethyl acetate, tetrahydrofuran, and acetone. Other non-solvents will readily be known to those of ordinary skill in the art. To be clear, the non-solvent (“second solvent”) is used to replace the solvent and cause the extraction of the first solvent such as NMP or DMSO, thereby causing the formation of the scaffold and to dissolve the pore forming agent, thereby producing pores in the scaffold.

The pore forming agents that may be used are particles of a desired size that are insoluble in the first solvent but that are soluble in the second solvent. The particles may be sized and present in sufficient concentration so as to create pores of a sufficient size to permit a plurality of mammalian cells to grow into and throughout the interconnected pores. The pore forming agents may be any of a variety of materials, depending on the particular selection of the solvent and non-solvent. Examples include: salt crystals such as NaCl, KCL, MgCl.sub.2, CaCl.sub.2 and BaSO.sub.4; soluble proteins such as albumin, globulins, and the like; soluble dextrans such as dextran and dextransulfates, and the like; soluble hydrogels such as agarose, alginate, chitosan, cellulose, carboxymethylcellulose, and the like; and microspheres of polylactic acid, polyglycolic acid, and the like. Those of ordinary skill in the art will readily be able to select useful pore forming agents. Tables 1, 2, and 3 in the Examples provided in Valentini are examples of the use of different sizes and concentrations of NaCl as well as various lyophilization techniques to produce a variety of pore sizes and shapes.

U.S. Pat. No. 5,977,204 to Boyan et al. (1999), which is expressly incorporated by reference herein, describes biodegradable polymeric therapeutic implantable materials that may be used to form the present biodegradable conduits. In this regard, polymers known in the art for producing biodegradable implant materials include polyglycolide (PGA), copolymers of glycolide such as glycolide/L-lactide copolymers (PGA/PLLA), glycolide/trimethylene carbonate copolymers (PGA/TMC), polylactides (PLA), stereocopolymers of PLA such as poly-L-lactide (PLLA), Poly-DL-lactide (PDLLA), L-lactide/DL-lactide copolymers, copolymers of PLA such as lactide/tetramethylglycolide copolymers, lactide/trimethylene carbonate copolymers, lactide/.delta.-valerolactone copolymers, lactide .epsilon.-caprolactone copolymers, polydepsipeptides, PLA/polyethylene oxide copolymers, unsymmetrically 3,6-substituted poly-1,4-dioxane-2,5-diones, poly-.beta.-hydroxybutyrate (PHBA), PHBA/.beta.-hydroxyvalerate copolymers (PHBA/HVA), poly-.beta.-hydroxypropionate (PHPA), poly-p-dioxanone (PDS), poly-.delta.-valerolatone, poly-.epsilon.-caprolactone, methylmethacrylate-N-vinyl pyrrolidone copolymers, polyesteramides, polyesters of oxalic acid, polydihydropyrans, polyalkyl-2-cyanoacrylates, polyurethanes (PU), polyvinyl alcohol (PVA), polypeptides, poly-.beta.-maleic acid (PMLA), and poly-.beta.-alkanoic acids.

Other biodegradable polymers that may be used to make the present biodegradable conduits are known in the art and include aliphatic polyesters, polymers of polylactic acid (PLA), polyglycolic acid (PGA) and mixtures and copolymers thereof, 50:50 to 85:15 copolymers of D,L-PLA/PGA, and 55/45 to 75:25 D,L-PLA/PGA copolymers. Single enantiomers of PLA may also be used, such as L-PLA, either alone or in combination with PGA. These polymeric implant materials may have a molecular weight of between about 25,000 and about 1,000,000 Daltons; between about 40,000 and about 400,000 Daltons; and between about 55,000 and about 200,000 Daltons. These polymeric implant materials may be capable of maintaining a pH of between about 6 and about 9 in a physiological environment. In another embodiment, they may maintain a pH of between about 6.5 and about 8.5

The preparation of precipitated polymers is well-known in the art. In general, the process comprises mixing a dried polymer mix with an art-known solvent such as acetone, methylene chloride or chloroform (e.g., acetone); precipitating the polymer mass from solution with a non-solvent, e.g., ethanol, methanol, ether or water; extracting the solvent and precipitating agent from the mass until it is a coherent mass that can be rolled or pressed or extruded into a mold; and curing the composition to the desired shape and stiffness. Further, porous composite materials that may be used for the present biodegradable conduits may be made as described in U.S. Pat. No. 5,863,297 to Walter et al. (1999), which is expressly incorporated herein by reference, with bioactive ceramic added to the polymer before curing.

Porous implant materials disclosed in Boyan and useful for making the present biodegradable conduits may have a porosity between about 60 and 90 volume percent, wherein the pore size distribution throughout the material is substantially uniform. The porosity may be achieved by adding more or less polymer to the mold. The porous implant materials disclosed in Boyan and useful for making the present biodegradable conduits may have an average pore size of between about 5 μm and about 400 μm. In another embodiment, the average pore size may be between about 100 μm and about 200 μm. Porous materials may contain no more than about 40 volume percent Bioglass.RTM. ceramic. In another embodiment, the porous materials may contain no more than about 20 to 30 volume percent Bioglass.RTM.ceramic. Nonporous implants may contain up to about 70 volume percent Bioglass.RTM.ceramic.

The implant materials disclosed in Boyan and useful for making the present biodegradable conduits may include channels to facilitate tissue ingrowth, and may be infiltrated with nutrient and/or cellular material such as blood and narrow cells, cartilage cells, perichondrial cells, periosteal cells, and other cells of mesenchymal origin, e.g., osteoblasts, chondrocytes, and their progenitors, adipocytes, muscle cells, tendon cells, ligament cells, dermal cells and fibroblasts, to facilitate tissue growth. The implant materials disclosed in Boyan and useful for making the present biodegradable conduits may also incorporate bioactive agents such as enzymes, growth factors, degradation agents, antibiotics and the like, designed for release over time.

To further biocompatibility, the implant materials disclosed in Boyan and useful for making the present biodegradable conduits may be substantially free of solvent. It is recognized that some residual solvent will be left in the polymer, but preferably less than about 100 ppm.

As is known to the art, the lifetime of the material in vivo may be increased by increasing the amount of D,L-PLA or L-PLA content, molecular weight and degree of crystallinity; or decreased by decreasing the same factors. The addition of bioactive ceramics may also decrease the molecular weight, and therefore decrease the degradation period.

To make the polymer/bioactive ceramic compositions disclosed in Boyan and useful for making the present biodegradable conduits a suitable polymeric material is selected, taking into consideration the degradation time desired for the implant material. Selection of such polymeric materials is known in the art. For example, PLA is used when a lengthy degradation time is desired, e.g., up to about two years. For a low target molecular weight, e.g., around 20,000 Daltons, 50:50 or 55:45 PLA:PGA copolymer is used when an approximately two-week degradation time is desired. To ensure a selected target molecular weight, degradation time, the molecular weights and compositions may be varied as known in the art depending on the mass of the implant formed from the polymer/bioactive ceramic composition.

The degradation of PLA and PGA has been extensively studied, both in vivo and in vitro. A number of factors affect the degradation rate of PLA:PGA copolymers, such as molecular weight, copolymer ratio, polymer crystallinity, thermal history, shape and porosity, and wettability. Additionally, other factors such as anatomical site of the implant, vascularity, tissue interaction and patient response affect the degradation rate in vivo. Depending on the above listed factors, degradation times for PLA and PGA polymers have been reported as low as 7 days for 50:50 PLG, to several years for PLA. The overall degradation kinetics have been fairly well established for the entire family of homopolymers and copolymers. Table 3, below, summarizes the findings of the degradation rates of the copolymers. Since this table is a compilation of many studies, the broad degradation range is reflective of the different experimental variables and parameters utilized.

TABLE 3
Degradation Rates of Polymers

| PLA/PGA | Degradation Time |
|---|---|
| 100/0 | 24 weeks-over 4 years |
| 85/15 | 12-34 weeks |
| 75/25 | 4-20 weeks |
| 70/30 | 25-30 weeks |
| 50/50 | 1-8 weeks |
| 0/100 | 8-20 weeks |

Implant materials disclosed in Boyan and useful for making the present biodegradable conduits may have a glass transition temperature (Tg) between about 38° C. and about 50° C. The incorporation of bioactive ceramics decreases the glass transition temperature of porous composite materials.

V. METHODS OF TREATMENT

A. Nerve Tissue Damage

The present invention envisions a variety of different clinical settings in which the disclosed methods and devices could be utilized. As discussed previously, nerve pathology arises from a wide variety of problems, including mechanical, thermal, or electrical trauma, congenital defects, or acquired disease states. In each of these settings, damage to nerve tissue may reduce or eliminate proper nerve cell contacts, and thereby reduce or eliminate transmission of signals to and from distal sites.

Acquired pathologies can be grouped into a variety of general categories. For example, cranial neuropathies include olfactory (trauma, olfactory groove meningioma), optic (otic neurits, Leber's disease, optic nerve glioma, ischemic optic neuropathy), oculomotor oculomotor (trauma, microvessel ischemia, compression), trochlear (trauma, microvessel ischemia, compression), trigeminal (trigeminal neuralgia, scleroderma), abducens (trauma, raised intracranial pressure, microvessel ischemia, compression), facial, (Bell's palsy, Lyme disease, sarcoidosis), vestibulocochlear (vestibular neuronitis, acoustic schwannoma), glossopharyngeal (glossopharyngeal neuralgia, motor neuron disease, tumor), vagus (motor neuron disease, tumor), accessory (trauma, tumor) and hypoglossal (motor neuron disease, tumor).

Inherited neuropathies include Charcot-Marie-Tooth disease type 1, PMP22 mutation, connexin-32 mutation, Po gene mutation, Werdnig-Hoffman disease, Kennedy syndrome, defect in androgen receptor gene, diseases related to motor neurons, diseases related to neuromuscular junctions, spinal cord diseases and CNS disorders.

Drugs that can cause polyneuropathies include antineoplastics (cisplatin, taxoids, vinca alkyloids), antiretrovirals (didanosine, stavdine, zalcitabine), antimicrobials (chloramphenicol, dapsone, isoniazid, metronidazole, nitrofurantoin), rheumatologic drugs (chloroquine, colchicine, gold, thalidomide) and a variety of others (amiodarone, disulfiram, perhexilene, phenytoin, pyridoxine, simvastatin). Chemicals that can cause polyneuropathies include acrylamide, allyl chloride, carbon disulfide, demethylaminopropionitrile, ethylene oxide, hexane, methyl bromide, methyl butyl ketone, organophosphorous esters, polychlorinated biphenyls, tricholoroethylene and vacor).

B. Surgical Procedures

Traditional nerve grafts involve the ability of harvesting sensory donor nerves from one location and placing them within a defect and bridging nerve gaps. The nerve cables, as they are called, are placed within these nerve defects using microsurgical techniques and very small 9-0 or 10-0 nylon sutures. Sutures are passed through the outer layer of the nerve and the nerve grafts in order for axons to subsequently travel down these bridging nerve cables. A similar approach can be employed according to the present invention wherein conduits are placed much like the nerve cables.

C. Induction of Growth Factor Expression

In an important aspect, the current invention contemplates the use of inducers of gene expression systems to “turn on” the production of nerve stimulating growth hormones in the helper cells. As discussed above, a number of genetic elements are available which can be selectively induced by exogenous factors. One issue, therefore, is how to deliver the exogenous inducer to the device in vivo.

At least two methods appear feasible for introduction of exogenous inducers. First, one may simply provide a catheter that terminates at or near (locally, regionally) the location of the device. Catheterization has the added advantage of permitting removal of excess fluid surrounding an implanted device. Another possible mechanism is through intravenous delivery. Preliminary studies have show delivery of an exogenous agent to an implanted device using distal, intravenous introduction. In addition, one can envision a combination of these two approaches, where catheterization of local or regional vasculature will permit effective delivery of an agent.

Post-operative catheter technology includes the placement of drains into surgical wounds that would be removed as the amount of fluid within the operative site decreases. These drains are traditionally composed of flexible silicone or alternative compounds and are easily removed after their adherence to the skin is severed. These drains also can be used in a reverse manner to induce agents to help regulate the delivery of growth factors.

X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Poly (L-Lactic acid)(PLLA) Biodegradable Nerve Scaffolds for Peripheral Nerve Regeneration

Material and Methods

In Vitro Trials

Conduits. Conduits were manufactured by a previously outlined technique.¹⁻³ Briefly poly (L-Lactic acid) (PLLA)(Birmingham Polymer, Birmingham, Ala.) was dissolved in methylene chloride and salt crystals (150-300 μm) were added to the polymer solution. The formed suspension was allowed to evaporate and the resulting PLLA/salt composite disks were cut, placed into a piston extrusion tool (Model 3912, Carves Inc. Wabash, Ind.), and heated at a rate of 25° C./min using a band heater (Watlow, St. Louis, Mo.).¹⁻² The temperature was allowed to equilibrate for 8 min and then the PLLA/salt composite was extruded (10 mm/s) to form a tube with an inner diameter of 1.6 mm and an outer diameter of 3.2 mm. The tubes were cut to 12 mm lengths (diamond wheel saw—Model 650, South Bay Technology, San Clemente, Calif.) and underwent a salt leaching and vacuum drying process.

Manufacture Characteristics. PLLA was studied to analyze the effects of the salt weight fraction, salt particle size and processing temperature on porosity and pore size of the extruded conduits by mercury intrusion porosimetry (Autoscan-500, Wuantachrome, Synosset, N.Y.).² Mechanical testing was performed by placing the ends of 30 mm long extruded conduits into aluminum tubes using paraffin (Vitrodyne V-100, Chatillon, Greensboro, N.C.). Tensile properties of the conduits were measured by pulling the conduits at a speed of 0.1 mm/s.² The influence of degradation on the mechanical properties were evaluated by maintaining the embedded and pre-wet conduits in PBS solution at 37° C. for 8 weeks. The molecular weight was determined by gel permeation chromatography as previously described.²

In Vivo Trials

Surgical Technique. Twenty-one Sprague Dawley (250 gms) rats with a right sciatic nerve defect were implanted with the 12 mm PLLA conduits. An additional 10 animals received nerve isografts from 5 donor animals for control. Briefly the animals were anesthetized and maintained by a 0.4 cc intramuscular injection of a premixed solution containing 64 mg/ml ketamine HCL (Keta-Sthetic™ Boehringer Ingelheim, St Joseph, Mo.), 3.6 mg/ml xylazine (Rompun™ Miles Inc., Shawnee Mission, Kans.), and 0.07 mg/ml atropine sulfate (Elkins-Sinn Inc., Cherry Hill, N.J.). The skin from the clipped lateral thigh was scrubbed in a routine fashion with antiseptic solution. The incision extended from the greater trochanter to the midcalf distally. The sciatic and posterior tibial nerves were exposed by a muscle splitting incision. The sciatic nerve was divided near its origin to create an adequate distal segment. The 12 mm conduits were placed into this defect using 10-0 nylon sutures under microsurgical technique. The nerve was sutured into the conduit such that 1 mm of each nerve end remained within the tubular biodegradable scaffold. Muscle and skin were closed using 4-0 Dexon sutures.

Functional Assessment. Walking track analysis was performed on all animals after conduit placement monthly through 16 weeks. This time schedule was chosen to allow adequate time for nerve regeneration, considering the length of the nerve graft and distal segment. Changes in the SFI correlate with changes in the paw print and are indicative of nerve regeneration. Although the SFI is an indirect measurement, it has been well established as an indicator of functional nerve restoration.

The medical and lateral gastrocnemius muscle was harvested at 6 and 16 weeks and weighed in order to assess nerve reinnervation. The gastrocnemius muscle is supplied by the posterior tibial branch of the sciatic nerve. Once the nerve is severed, the muscle will begin to atrophy. As the nerve regenerates into the muscle, it will regain its mass proportional to the amount of reinnervation. This will provide indirect measurements of nerve regeneration. Weight was determined by placing the muscle in preweighed sterile saline containers so that dessication did not occur. The difference in weight of the containers before and after muscle placement determined the muscle weight.

Histological Assessment. At the conclusion of 6 and 16 weeks, sections of the conduit/grafted and distal nerve from the same rats used for functional evaluation were harvested, fixed with 3% glutaraldehyde, embedded in epoxy resin and stained with toluidine blue. The toluidine blue stained nerve sections were placed on the stage of an inverted microscope and viewed with phase optics. Images of the histological sections were digitized using a CCD camera and subsequently analyzed using standard image processing and analysis techniques. Images were thresholded and segmented into individual axons. The number of axons were counted to give the number of axons/mm². The area of each axon was determined, summed together, and expressed as area axons/image area to give the nerve fiber density.

Statistical Analysis. Statistical analysis was performed by two sampled T-tests for independent samples with adjustment of variance and Wilcoxon rank sum tests, where appropriate. Statistical analysis for functional parameters was based on mixed affects model using the maximum likelihood method.^{5,6} The model includes a random subject effect to reflect that rats are samples of a population and to induce correlation between measurements from the same rat. Since possible treatment differences are observed after the operation, the interaction effects in the model indicate the treatment effects assuming a common operation effect across all rats. To assess if the means of the sciatic functional index vary among groups of rats with different treatments, the model allows each rat to have a random initial index as well as a random average decline in an index over several months. To determine the mean number of axons and the mean nerve fiber density, the model includes treatment and location effects, their interaction effects, as well as a random subject effect.

Results

Mechanical Testing

The pressure necessary to extrude the tubular polymer/salt composite structures was highly dependent on the extrusion temperature. Increasing the salt fraction resulted in an increased conduit porosity. The pore diameter also increased as the salt weight fraction increased. The porosity however was not affected by the extrusion temperature. Scanning Electron Microscopy photomicrographs of the conduits revealed extensive pores which were roughly spherical in shape and evenly distributed throughout. Measured porosity was within 2% of the calculated values.

Tensile testing indicated that strength decreased with degradation during the first 2-4 weeks in phosphate buffered saline (PBS). These values then increased for up to 8 weeks then decreased again. This increase is believed to be secondary to swelling of the polymer.⁷⁻⁸ All conduits remained stable and did not change shape during the entire time course of degradation. The average molecular weight of the PLLA conduits was 35,500 (76% of the initial values). After 8 weeks in PBS, the molecular weight decreased to 43% of the day 0 value.

In Vivo Trials

Physical Assessment. All conduits remained flexible without breakage. No conduit elongated during the 4 months of placement. Proximal and distal nerve ends were noted to have maintained their adherence to the conduit through the use of the microsuture. No severe inflammatory reaction could be identified and no neuromas were apparent clinically.

Functional Assessment. Evaluation of the Sciatic Functional Index for both autogenous and conduit grafts demonstrated improved functional recovery monthly through 16 weeks noting muscle reinnervation. Autogenous nerve isografts were statistically lower than PLLA conduits by 16 weeks (p=0.0002, Std Dev=5.25). The weight of the gastrocnemius muscle demonstrated a greater statistically significant increase in the autogenous isografts than the PLLA conduits.

Histological Assessment. Histomorphology demonstrated axonal migration and nerve tissue advancement through the entire conduit and into the distal nerve stump at 6 and 16 weeks. However as expected, when compared to the autogenous isografts, axon number/mm² and nerve fiber density in the distal nerve was statistically less except for nerve fiber density at 16 weeks (p=0.0015, Std Dev=2534; p=0.0115, Std Dev 0.0918 respectively at 6 weeks; p=0.0039, Std Dev=3593, p=0.1027, Std Dev=0.06 at 16 weeks respectively).

When evaluating the axon number/mm² and the nerve fiber density in the midconduit, the autogenous isografts were greater than the conduits except at 16 weeks in which both were greater (p=0.000, Std Dev=3997, p=0.014, Std Dev=0.163 respectively at 6 weeks; p=0.0026, Std Dev=5162, p =0.0617, Std Dev=0.12 respectively at 16 weeks).

### EXAMPLE 2

Long-Term In Vivo Evaluation of Poly (L-Lactic Acid) Porous Conduits for Peripheral Nerve Regeneration

Material and Methods

Fabrication of the Polymer Conduit. Poly(L-lactic acid) (PLLA) (Birmingham Polymers, Birmingham, Ala.) were manufactured into porous biodegradable conduits using a combined solvent casting, extrusion, and particulate leaching technique. The number average molecular weight (Mn) of the raw material was 46,500±2,100 (n=5), as measured by gel permeation chromatography. The PLLA conduits were fabricated with a salt weight fraction of 90%, a salt crystal size between 150 and 300 μm, and an extrusion temperature of 275° C. The resulting conduits had an inner diameter of 1.6 mm, an outer diameter of 3.2 mm, and a length of 12 mm. The PLLA conduits had an interconnected pore structure, and the porosity and mean pore size were measured by mercury porosimetry as 83.5±4.1% and 12.1±2.8 μm, respectively. The Mn of the processed PLLA decreased to 35,500±2,700. Tensile testing indicated a strength of 81.7±35.1 MPa, a modulus of 1.0±0.4 MPa, and a strain at break of 0.02±0.01 mm/mm. The crystallinity of the PLLA conduits was 5.2±0.4%.

In Vivo Study. Thirty-one Sprague Dawley rats (200-250 g) (Harlan, Wis.) were utilized. Sprague Dawley animals were chosen due to match previous short-term studies with the PLLA conduits in our laboratory.⁵ The animals were randomly assigned to two groups. The control group of 10 animals received a 12 mm reversed isograft into the right sciatic nerve from 5 donor animals. The experimental group (n=21) received a 12 mm empty PLLA conduits placed into a 10 mm defect in the right sciatic nerve. The left leg served as an internal control. All animals were maintained in facilities approved by the American Association for Accreditation of Laboratory Animal Care and in accordance with current United States Department of Agriculture, Department of Health and Human Services, and National Institutes of Health regulations and standards.

Surgical Technique. Briefly the animals were anesthetized and maintained by a 0.4 cc intramuscular injection of a premixed solution containing 64 mg/ml ketamine HCL (Keta-Sthetic™ Boehringer Ingelheim, St Joseph, Mo.), 3.6 mg/ml xylazine (Rompun™ Miles Inc., Shawnee Mission, Kans.), and 0.07 mg/ml atropine sulfate (Elkins-Sinn Inc., Cherry Hill, N.J.). The skin from the clipped lateral thigh was scrubbed in a routine fashion with antiseptic solution. The incision extended from the greater trochanter to the midcalf distally. The sciatic nerve was exposed by a muscle splitting incision. The sciatic nerve was divided near its origin to create an adequate distal segment. The 12 mm PLLA conduits, or reversed isografts, were placed into this nerve gap using 10-0 nylon sutures (Sharp Point) under microsurgical technique. The nerve was sutured into the conduit such that 1 mm of each nerve end remained within the tubular biodegradable scaffold. Muscle and skin were closed using 4-0 Dexon sutures (Davis+Geck, Wayne, N.J.).

Functional and Histologic Evaluation. Walking track analysis was performed monthly through 8 months. Walking track analysis measures functional muscle reinnervation by comparing the correction of paw prints from the experimental limb relative to the contralateral control limbs. The sciatic functional index (SFI) has been previously described and expressed as an absolute value.^{4-7,9,10} Although the SFI is an indrect measurement, it has been well established as an indicator of functional nerve recovery.⁹⁻¹⁰ As the value tends toward zero, a better functional recovery is noted. Preoperatively, the animals were trained to walk down a 30+4 inch track into a darkened enclosure. Postoperatively, the rats' hind paws were painted with water soluble ink and any changes in their paw prints caused by nerve injury and denervation were recorded. Three different parameters of the rodent's paw print were measured to determine the sciatic functional index:⁹⁻¹⁰

Sciatic Functional Index=−38.3(PLF)+109.5(TSF)+13.3(ITF)−8.8

PLF=Print Length Function=(Experimental PL−Normal PL)/Normal PL

TSF=Toe Spread Function=(Experimental TS−Normal TS)/Normal TS(1st to 5th Toe)

ITF=Intermedian Toe Spread Function=(Experimental IT−Normal IT)/Normal IT(2nd to 4th Toe)

At the end of 4 months, 3 animals in the control isograft group and 6 animals in the experimental group had the medial and lateral gastrocnemius muscles harvested and weighed for comparison. The gastrocnemius muscle is supplied by the posterior tibial branch of the sciatic nerve. Once the sciatic nerve is severed, the muscle will begin to atrophy. As the nerve regenerates, the muscle will regain its mass proportional to the amount of reinnervation. This provides indirect measurements of nerve regeneration. Muscles were placed in pre-weighed sterile containers containing saline to prevent dehydration. The difference in mass of the containers before and after muscle placement determined the muscle mass.

Following muscle evaluation, the midconduit/isograft and the distal nerve in these same animals were harvested and histomorphologically analyzed. Conduits were also measured and compared to their preimplantation length to determine if elongation had occurred. The midconduit/isograft was fixed with 3% glutaraldehyde, embedded in epoxy resin, and stained with toluidine blue. Toluidine blue stains axon myelin and does not react with PLLA. Stained midconduit/isograft and distal nerve sections were placed on the stage of an inverted microscope and viewed with brightfield optics. Images of the histological sections were digitized using a CCD camera and analyzed. Briefly, images were thresholded and segmented into individual axons. The number of axons were counted to give the number of axons/image area, and the area of each axon was determined, summed, and expressed as axons area/image area to give the nerve fiber density.

Similar studies were performed on the remaining 14 animals (5 control and 9 experimental) at 8 months. Two animals from the control isograft group and 6 animals from the experimental group could not be evaluated due to anesthetic deaths or euthanasia due to self mutilation.

Statistical Analyses. Multiple samples were collected and expressed as means±standard error. A two sample T-test and Wilcoxon rank sum test was used to compare the variables between isograft and conduit groups for histomorphology and gastrocnemius muscle weights. Significance level was set at α=0.05. After Bonferroni correction for multiple testing, a p value of ≦0.01 was considered statistically significant. Two-sample t test and Wilcoxon Rank Sum test were used to compare the SFI between isograft and conduit groups. Significance was set at α=0.05.

Results

Throughout all time periods, the PLLA conduit remained structurally intact. There was tissue incorporation and vascularization. There was no evidence of conduit collapse or breakage with limb ambulation. Moreover there was no evidence of conduit collapse or elongation at 8 months as observed with the 75:25 Poly(DL-lactic-co-gylcolic acid)(PLGA) conduits.

Functional and Histolomorphologic Evaluation. The mean absolute value of the SFI demonstrated no group differences between the isograft controls and experimental animals measured over the 8 months except at 3 months where the isograft values were higher (p=0.0379) and at 7 months were the isograft group was significantly lower (p=0.0115).

At 4 months, the weight of the gastrocnemius muscles in the experimental animals was not significantly different from isografts, although there was a trend for more muscle mass in the isograft group (p=0.3662). By 8 months this comparison and trend remained (p=0.0619).

At 4 months the number of axons/mm² and nerve fiber density was not significantly different between the isograft and conduit groups in either the midconduit/isograft or distal nerve.

At 8 months the number of axons/mm² was significantly lower in the isograft controls compared to the midconduit (p=0.006). The number of axons/mm² in the distal nerve and the nerve fiber density in the midconduit and distal nerve were not significantly different between the two groups.

### EXAMPLE 3

Dermal Fibroblast Genetically Engineered to Release Nerve Growth Factor

Materials and Methods

Isolation of Rat β-NGF cDNA. Rat β-NGF was isolated from rat SCs by RT-PCR. PCR was performed with primer 1 (5′-atataagcttcatccacccacccagtc-3′) and primer II (5′-atataggatcctcatcttgcagcttccctg-3′) using mutants as templates to introduce HindIII and XbaI sites at both ends of the cDNA encoding rat β-NGF. The absence of nucleotide misincorporation during PCR was checked by sequencing. PCR amplication (Perkin Elmer GeneAmp PCR System 2400) was conducted at 94° C. for 30 s during denaturation, 55° C. during 30 s primer annealing, and 72° C. for 30 s during primer extension.

Construction of Expression Vecotrs. Rat β-NGF cDNA fragments were digested by HindIII and XbaI (Biolabs) and ligated with the vector pSec TagHygro (Invitrogen). The vector pZeoSVLac (Invitrogen) was utilized to insert the reporter gene LacZ and assess transfection efficiency using β-Gal.

Transfection. Rat DFB's were seeded at 4×10⁴ cells/well in 6-well plates (Falcon) 18 hrs prior to transfection. Immediately prior to transfection, DFB's were rinsed and refed FBS-free DMEM. For indirect lipid-based transfection, the reagents Transfectam (Promega), ESCORT (Sigma), DOTAP (Boehringer Mannheim), and FuGENE6 (Boehringer Mannheim) were used per manufacturer's instructions. In a few experiments, direct transfection was accomplished using a gene gun. Briefly, plasmid DNA was coated onto Au particles (0.6 μm, BioRad) and shot into DFB's at either 100 psi (low pressure) or 200 psi (high pressure).

Transfection efficiency experiments were conducted in duplicate, and efficiency (mean±SEM) was expressed as (# positive β-Gal stained cells/total # cells)×100. β-Gal staining was conducted by first washing the DFB's post-transfection with PBS followed by fixation in 0.05% glutaraldehyde for 5 min at 25° C. After rinsing with PBS, the fixed DFB's were exposed to a staining solutions consisting of 20 mM K₃Fe(CN)₆, 2 nM MgCl₂, and 1 mg/mL, 5-bromo-4-chloro-3-indolyl-β-D-galactopyranoside for 2 hrs at 37° C. After elapseed time, the stained DFB's were rinsed with PBS and maintained in 2% paraformaldehyde. Stained/unstained DFB's were visualized and counted using brightfield microscopy (Olympus) coupled with digital image acquisition and analysis (IPLab).

NGF transfection experiments were conducted in quadruplicate, and NGF expression (mean±SEM) was expressed as either pg/mL, or normalized as pg/mL/cell. Briefly, DFB's were transfected using FuGENE6 for 30 min, rinsed and refed with 1 mL culture media, and allowed to secrete NGF for 24, 48 and 72 hrs. NGF concentrations in DFB supernatants were determined via NGF ELISA (Promega) per manufacturer's instructions. DFB number was determined by trypsinizing DFB monolayers and counting the resulting cell suspension with a Coulter counter.

Cell Culture. SCs were harvested from neonatal Sprage-Dawley rats following anesthesia and euthanasia (decapitation). Harvest and epineural dissection were performed utilizing a stero dissecting microscope (Olympus SZH 10). Fascicles were cut and placed on 35 mm dishes prepared with collagen I (Vitrogen 100) and Dulbecco's minimal essential medium (DMEM) supplemented with 10% fetal bovine serum (FBS, Gibco). Cultures were maintained for approximately ten days before confluent monolayers were generated.

DFB's were obtained from ATCC (CRL-1213). The cell line was developed from a skip biopsy of a fetal (18 day), germ free Sprague Dawley rat. DFB's were maintained in DMEM supplemented with 10% FBS. DFB's were passaged at 1:4 once 99% confluency was obtained.

Data Interpretation. Statistical significance was calculated using One-Way Analysis of Variance (ANOVA) with Student's t test. Statistical significance was defined as p≦0.05. NGF release is defined as the concentration of NGF (pg/ml or pg/ML/10³ cells) measured in media supernatants. Release rate is defined as the mass of NGF released over time (ng/10⁶ cells/day) measured in media supernatants.

Results

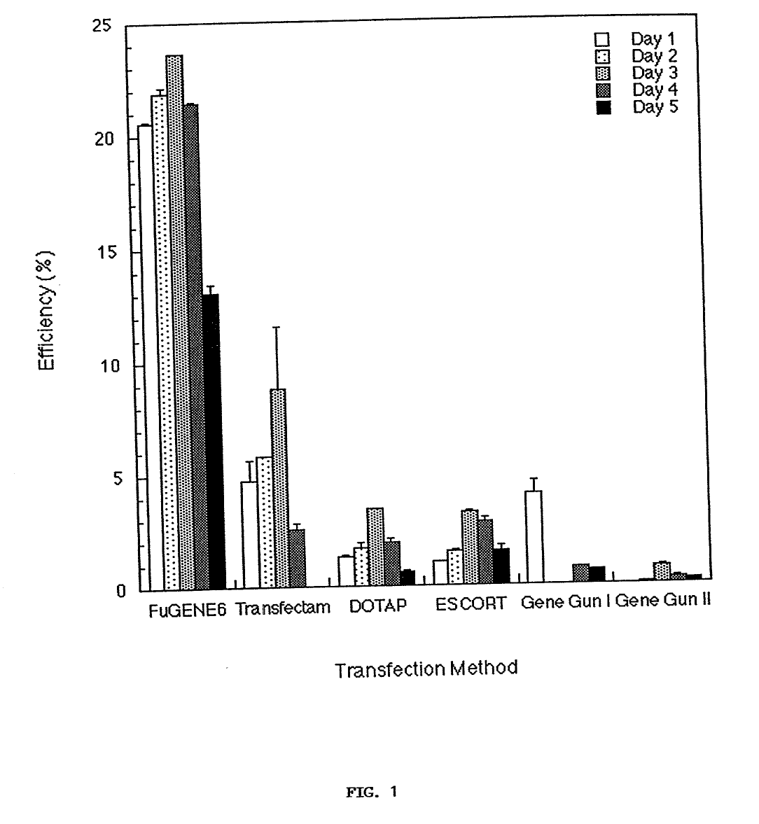

Transfection Method. To determine the optimum transfection method to be utilized, several direct and indirect methods were assessed using in vitro LacZ expression with β-Gal catalytic activity as an endpoint. For each method, transfection efficiency was assessed daily for up to five days to determine the temporal characteristics. As show in FIG. 1, the lipid-based transfection method utilizing FuGENE6 outperformed all remaining indirect and all direct transfection methods. Transfection efficiency using FuGENE6 ranged from 13% to 24% over a five-day period, with a five-day maximum and temporal average of 24% and 20.1±1.9%, respectively. FuGENE6 was utilized fro all subsequent NGF transfection experiments.

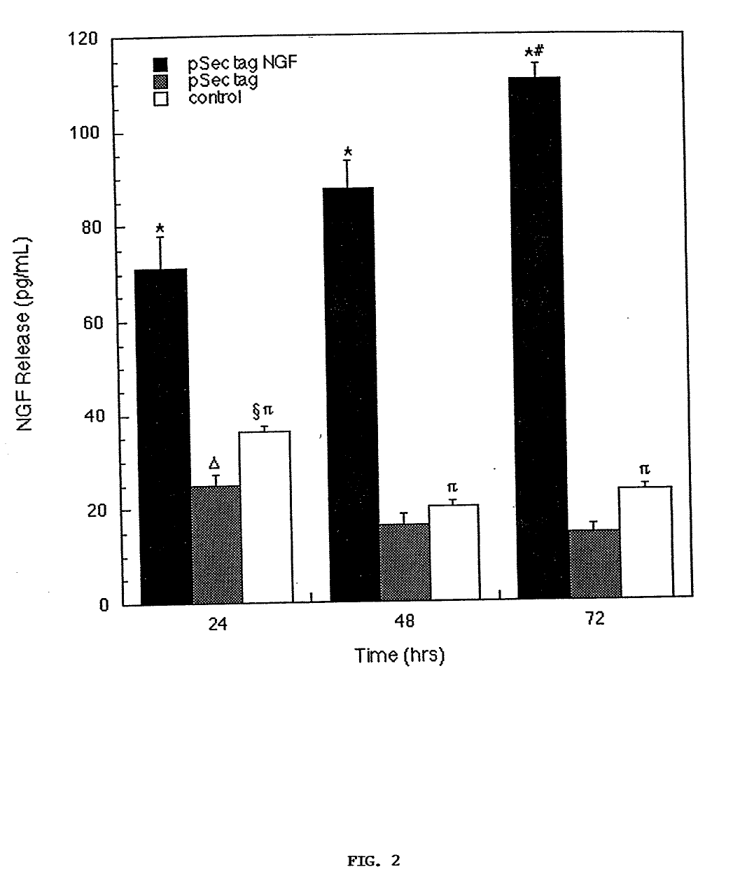

In Vitro NGF Release. The NGF release was determined from transfected or control DFB's over a three day period. As depicted in FIG. 2, NGF release from NGF transfected DFB's steadily increased to a value of 111 pg NGF/mL, with 72 hrs results being significantly different than both 24 and 48 hrs (p≦0.002 and p≦0.01, respectively). NGF release from NGF transfected DFB's was markedly higher than control DFB's or DFB's transfected with vector alone (p≦0.002). However, these data can be misinterpreted due to the fact that differences in cell number among replicates and with time (i.e., increasing cell number with culture time) exist.

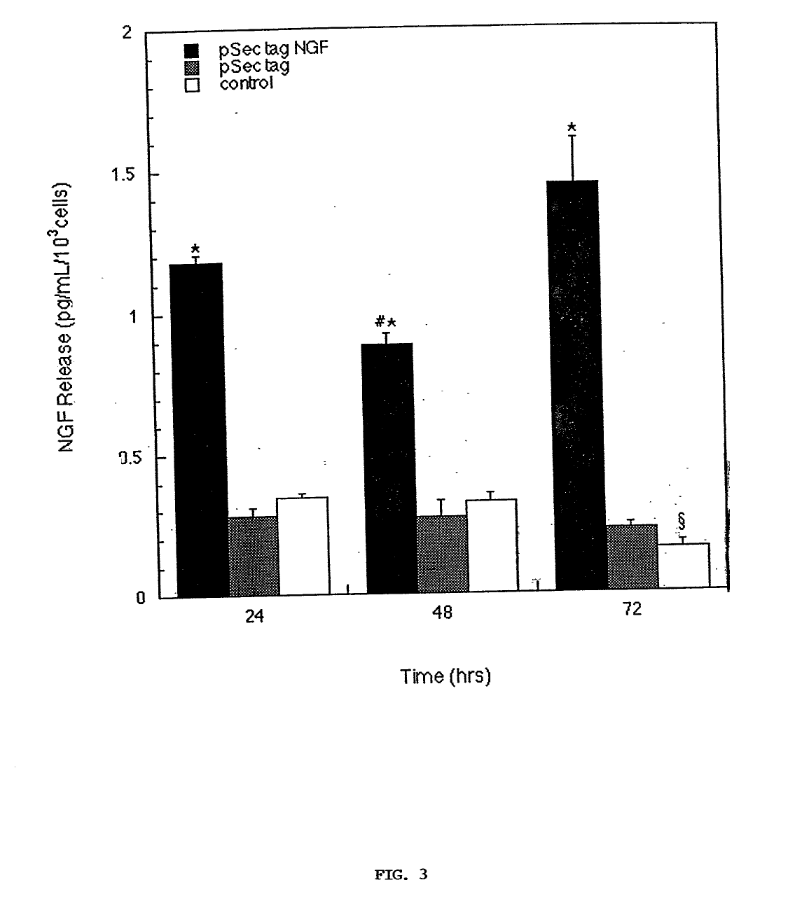

To remove the confounding problem of increasing DFB number with increased time, NGF released by transfected DFB's was normalized to cell number (FIG. 3). This results in a maximal release of 1.2 pg NGF/mL/10³ cells from NGF transfected DFB's at 72 hours. For all time periods, DFB's transfected with rat NGF expressed markedly higher levels of NGF as compared to control DFB's or DFB's transfected with vector alone. Interestingly, there was a statistically significant decrease (p≦0.01) in NGF released from NGF transfected DFB's at 48 hrs. This decrease was not observed with control or vector transfected DFB's. The same behavior was observed at 48 hrs with a human fibroblast cell line transfected with NGF (data not shown).

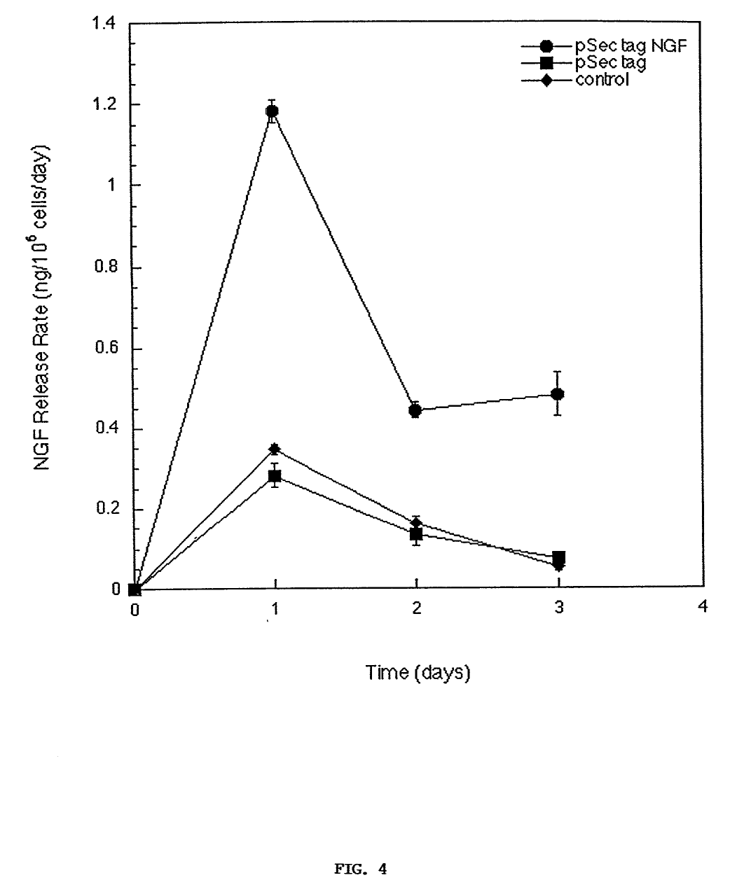

Knowing the volume of culture media, FIG. 3 was converted to a release rate curve (FIG. 4). The NGF transfected DFB's demonstrated maximal NGF release at day 1 (1.2 ng NGF/10⁶ cells/day), followed by a markedly lower, sustained release rate at days 2 and 3 (0.44 ng NGF/10⁶ cells/day and 0.48 ng NGF/10⁶ cells/day, respectively). Interestingly, the release rate curves for control and vector transfected DFB's also exhibited a maximal NGF release at day 1, but were followed by a decreasing release rate. These two release rate curves could potentially represent the NGF present in the FBS followed by NGF's in vitro degradation. The release rate curve of NGF transfected DFB's is significantly different than curves from control and vector transfected DFB's (p≦0.001).

### EXAMPLE 4

Muristerone A-Induced Nerve Growth Factor Release From Genetically Engineered Dermal Fibroblasts

Materials and Methods

Cell Culture. A hDFB cell line (NR6) was obtained from Dr. Zhen Fan, The University of Texas M.D. Anderson Cancer Center. hDFBs were maintained in DMEM-HG supplemented with 10% FBS (Sigma) and penicillin-streptomycin-glutamine mixture (Gibco). Medium for transfected hDFBs also was supplemented with 200 pg/mL Zeocin (Invitrogen) and 150 pg/mL 6418 (Invitrogen). hDFBs were passaged 1:4 once 90% confluency was obtained. For the NGF bioactivity assay, hDFBs were maintained in PC-12 culture medium and were seeded at 10⁵ cells/750 μL in 12-well plates.

PC-12 cells were obtained from ATCC (CRL-1721). PC-12 cells were maintained in F12k supplemented with 15% horse serum (Sigma, St. Louis, Mo.), 2.5% FBS, and penicillin-streptomycin-glutamine mixture (Gibco). For the NGF bioactivity assay, PC-12 cells were seeded at 10² cells/750 μL in 12-well plates.

Isolation of human NGF cDNA. Human NGF cDNA was amplified by PCR™ from a NGF encoding plasmid using the primers:

(1) 5′-ATAAGCTTGCCGCCATGTCCATGTTGTTCTA-3′
(2) 5′-TATATCTAGATCTCACAGCCTTCCTGC-3′

The PCR™ fragment was digested with HindIII and XbaI and inserted in a HindIII/XbaI digested vector pIND using the restriction sites included in the primers and the polylinker of the vector the resuting plasmid was named pIND-NGF. Correct insertion was confirmed by restriction analysis and sequencing.

Transfection. pIND-NGF and pVgRXR (regulator vector, Invitrogen) were then cotransfected into hDFBs in FBS-free DMEM-HG using FuGENE6 (Boehringer Mannheim) per manufacturer's instructions. Stable clones were selected using a combination of Zeocin (200 μg/mL) and 6418 (150 μg/mL) for pVgRXR and pIND), respectively. Untransfected and uninduced transfected hDFBs served as a negative control in all in vivo and in vitro studies. Muristerone A (3 μM) was used as the inducing agent for all in vitro and in vivo studies. Muristerone A is inert to mammalian physiology and thereby exerts no pleiotropic effects. In contrast to the manufactuer's instruction, Muristerone A was dissolved in DMSO and not in ethanol.

NGF Secretion. NGF concentrations in hDFB in vitro supernatants and in vitro chamber fluid were determined via NGF ELISA (Promega) per manufacturer's instructions. NGF release is defined as the concentration of NGF (μg/mL or μg/mL/10³ cells) measured. Release rate is defined as the mass of NGF released over time (ng/10⁶ cells/day) measured. hDFB number was determined by trypsinizing hDFB monolayers and counting the resulting cell suspension with a Coulter counter.

NGF Bioactivity. Bioactivity assay of released NGF was assessed using PC-12 cells with their differentiation as the endpoint. PC-12 cells were incubated with PC.12 culture media (control) or media supernatants from transfected hDFBs in the presence of MurA (i.e., media with released NGF). Briefly, hDFBs were cultured and exposed to 3 μM Muristerone for 3 days in 12-well plates.

The media, theoretically with containing NGF released from transfected hDFBs, was then removed and used to culture PC-12 cells in 12-well plates. After 4 days, random images were digitized of each well and the number of differentiated and undifferentiated Data are reported as fraction of PC-12 cells that were differentiated.

In Vivo NGF Secretion. NGF collection chambers were implanted subcutaneously in male nude rats (RNU/RNU, Harlan) under anesthesia (0.2 mL/100 gbw intramuscular injection of premixed solution composed of 64 mg/mL ketamine HCl, 3.6 mg/mL xylazine, and 0.07 mg/mL atropine sulfate). The rats had an average mass of 87 g (range 57-115 g) and were 7-8 weeks old. A nude strain is required to avoid an immune response to hDFBs placed in the collection chambers. The in vivo study was approved by the University of Texas M.D. Anderson Cancer Center Animal Care and Use Committee and in accordance with AAALAC and NIH guidelines. Collection chambers consisted of 14 mm silicone tubes cut to length from extension tubes possessing a 3.7 mm outer diameter and 0.5 mm wall thickness (Pharmaseal, K52).

After shaving the back, a longitudinal incision (˜2 cm each) was made through the skin of the right and left flank. Individual “pockets” for each collection chamber were prepared in the subcutaneous space of both flanks by blunt dissection. Collection chambers were inserted into each pocket and capped by suturing muscle to each end with 4-0 Prolene suture (Ethicon). The sutured muscle caps provided both a tight seal for the collection chamber and a vascular supply to maintain the viability of injected hDFBs. The sealed colleciton chambers were filled with either transfected hDFBs with Muristerone A (TFB/MurA (+)), transfected hDFBs without Muristerone A (TFB/MurA (−)), untransfected hDFBs (NTFB), or phosphate buffered saline (PBS) using a 1 cc tuberculin syringe with 27 G needle (Becton Dickinson). All transfected and untransfected hDFBs were injected at 10⁶ cells/mL. The incisions were closed with staples. Animals were housed individually and fed standard rat chow. The collection chambers were left in vivo one and two days. After the elapsed time, the rats were euthanized with CO₂ and the fluid within the collection chamber withdrawn with a 1 cc tuberculin syringe with 27 G needle (Becton Dickinson) and placed in a Eppendorf tube for subsequent NGF ELISA.

For this study, a total of 20 rats were used at 10 rats/time period and 5 rats/group. Each rat was implanted with two collection chambers. For each time period, 5 rats possessed collection chambers with TFB/MurA(+) and TFB/Mur(−) in the left and right chambers, respectively, and 5 rats possessed collection chambers with NTFB and PBS in the left and right chambers, respectively. The inducible agent, Muristerone A, was added to the hDFB suspensions (TFB/MurA (+) group) prior to injection into collection chambers. A total of 40 collection chambers were used in this study.

Data Interpretation. Statistical significance was calculated using One-Way Analysis of Variance (ANOVA) with Student's t test. Statistical significance was defined as p≦0.05.

Results

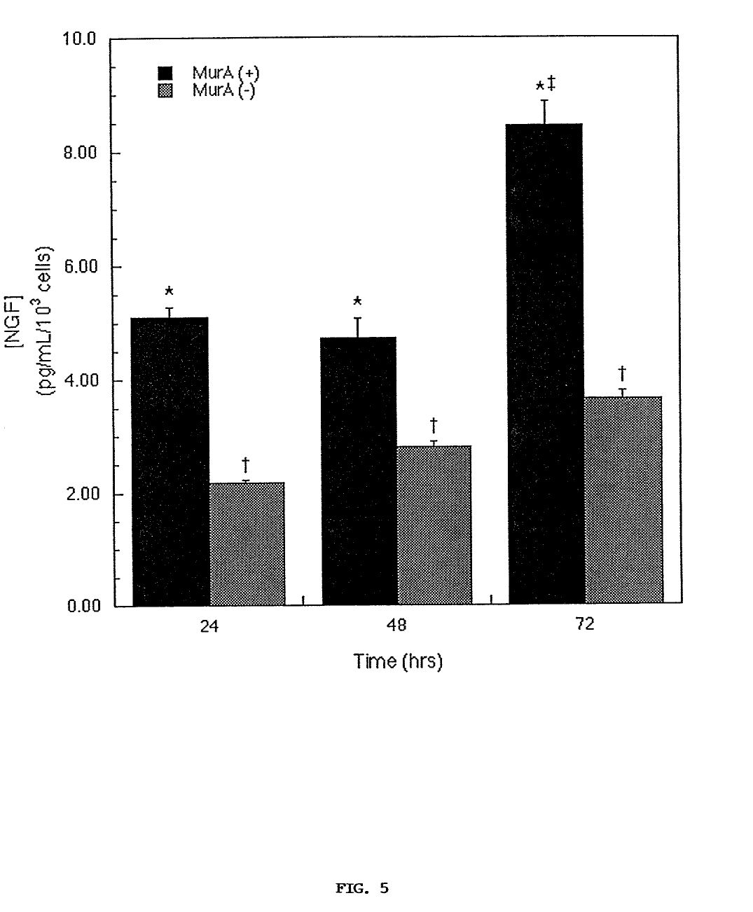

In Vitro NGF Release. The NGF release was determined from transfected hDFBs with (MurA (+)) and without (MurA (−)) the inducing agent Muristerone A over a three day period. To remove the potential confounding problem of increasing hDFB number with increased culture time, NGF released by transfected hDFBs was normalized to cell number (FIG. 5). This results in a maximal release of 8.5±0.4 pg NGF/mL/10³ cells from MurA (+) hDFBs at 72 h. For all time periods, MurA (+) hDFBs expressed markedly higher levels of NGF compared to MurA (−) hDFBs (p≦0.001). NGF release from MurA (+) hDFBs at 24 and 48 h was not signficantly different, whereas NGF release at 72 h was significantly different than either 24 or 48 h (p≦0.001). NGF release from MurA (−) hDFBs at all three timepoints were significantly different from each other (p≦0.001).

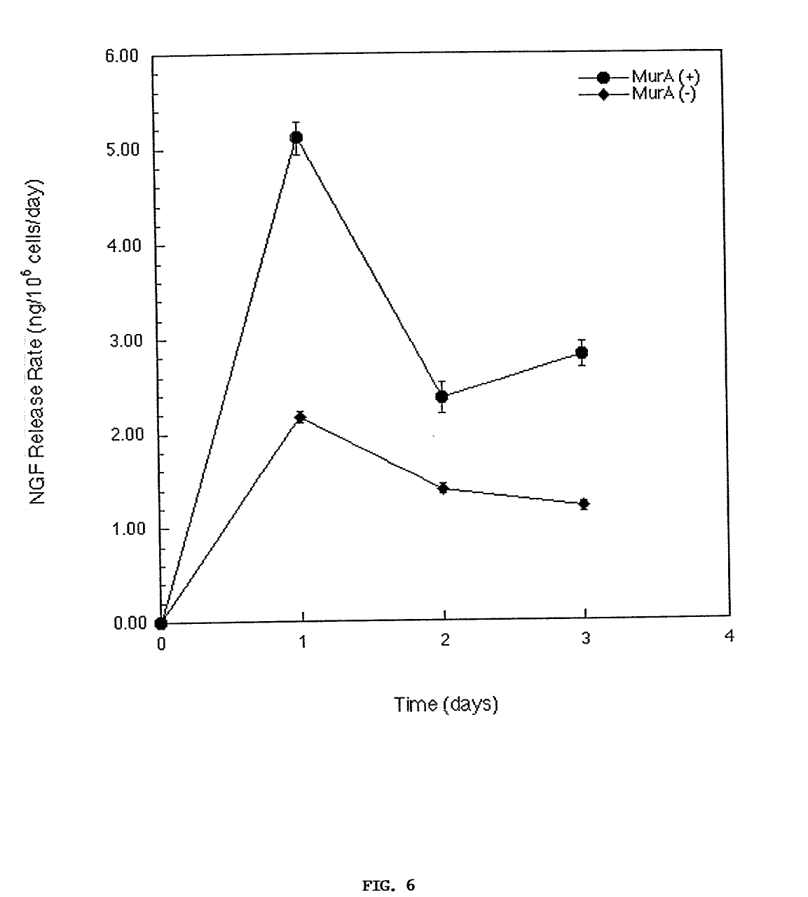

Knowing the volume of culture media, FIG. 5 was converted to a release rate curve (FIG. 6). MurA (+) hDFBs demonstrated maximal NGF release rate at day 1 (5.1±0.2 ng NGF/10⁶ cells/day), followed by a markedly lower, sustained release rate at days 2 and 3 (2.4±0.2 ng NGF/10⁶ cells/day and 2.8±0.1 ng NGF/10⁶ cells/day, respectively). Interestingly, the release rate curve for MurA (−) hDFBs also exhibited a maximal NGF release rate at day 1 (2.2±ng NGF/10⁶ cells/day), but was followed by a decreasing release rate. This release rate curves could potentially represent the NGF present in the FBS followed by NGF's in vitro degradation. The release rate curve of MurA (+) hDFBs is significantly different than curves from MurA (−) hDFBs (p≦0.001).

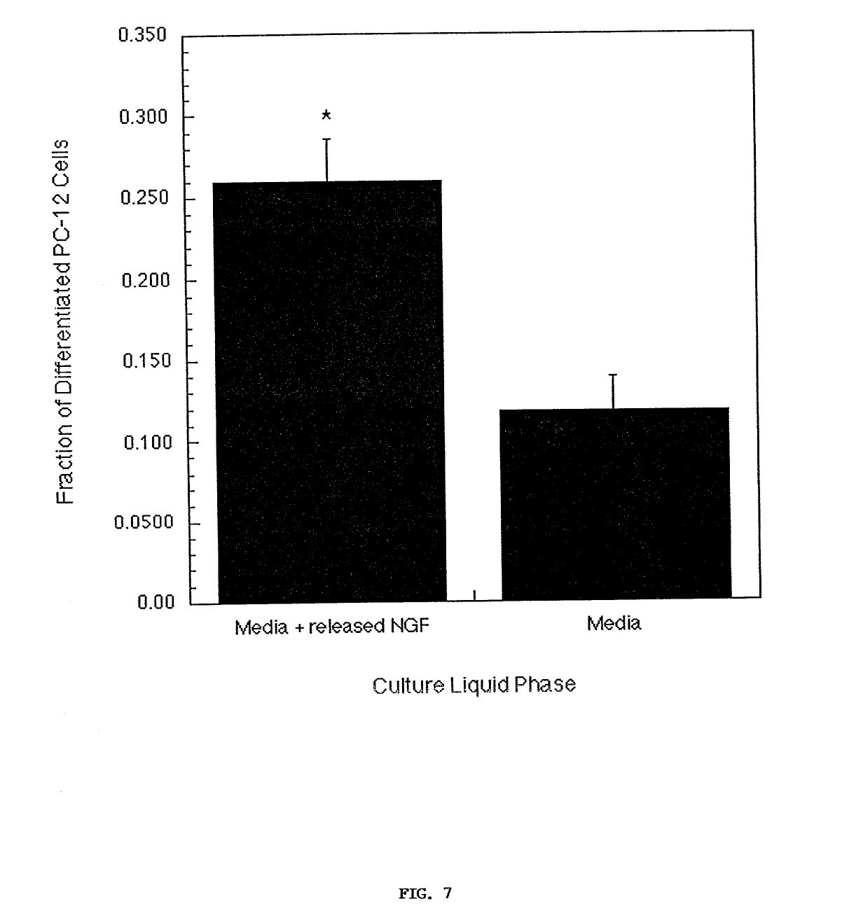

NGF Bioactivity. It is possible that NGF may be released from induced, transfected hDFBs in a non-bioactive form. An NGF ELISA only determines if the NGF epitope is present and can not allue to NGF bioactivity. Hence, PC-12 cells were used to assess bioactivity of secreted NGF with PC-12 differentiation being the measured endpoint. PC-12 cells does-dependently extend neurite-like processes in response to NGF. As shown in FIG. 7, PC-12 cells exposed to NGF secreted from hDFBs demonstrated markedly higher levels (p≦0.002) of differentiation compared to PC-12 cells exposed to media alone. PC-12 cells exposed to media do not exhibit 0% differentiation due to the small amount of NGF present in FBS.

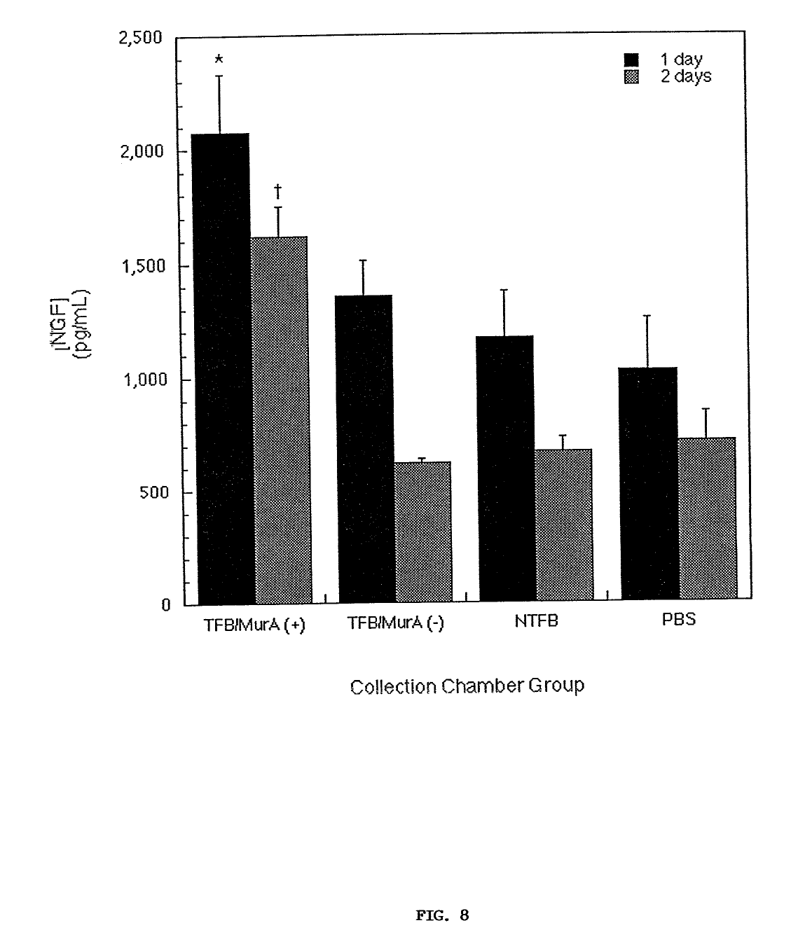

In Vivo NGF Release. NGF release was determined in vivo over a two day period using implanted collection chambers filled with induced, transfected hDFBs or various negative control groups. NGF concentration in collected chamber fluid was assessed via ELISA. As shown in FIG. 8, at both 1 and 2 days, TFB/MurA (+) possessed significantly higher NGF levels (2,074±257 pg/mL and 1,620±132 pg/mL, respectively) when compared to negative controls (p≦0.05 and p≦0.003, respectively). There was no statistical difference between the TFB/MurA (−), NTFB, and PBS groups at either day in keeping with the hypothesis that these three groups should yield wound fluid NGF levels. In agreement with the release kinetic trends of FIG. 6, the NGF released upon inducing transfected hDFBs was greatest at day 1 and decreased by 22% at day 2.

All of the compositions, methods and apparati disclosed and claimed herein can be made and executed without undue experimentation in light of the present disclosure. While the compositions and methods of this invention have been described in terms of preferred embodiments, it will be apparent to those of skill in the art that variations may be applied to the compositions, methods and apparati and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

EP Patent No. 216,453

WO Patent No. 95/24429

WO Patent No. 95/24497

WO Patent No. 95/04132

U.S. Pat. No. 4,683,202

U.S. Pat. No. 4,797,368

U.S. Pat. No. 5,139,941

U.S. Pat. No. 5,302,523

U.S. Pat. No. 5,464,765

U.S. Pat. No. 5,863,297

U.S. Pat. No. 5,925,565

U.S. Pat. No. 5,928,906

U.S. Pat. No. 5,935,819

U.S. Pat. No. 5,939.323

U.S. Pat. No. 5,977,204

Angel, Bauman, Stein, Dellus, Rahmsdorf, and Herrlich, “12-0-tetradecanoyl-phorbol-13-acetate Induction of the Human Collagenase Gene is Mediated by an Inducible Enhancer Element Located in the 5′ Flanking Region,” Mol. Cell. Biol., 7:2256, 1987a.

Angel, Imagawa, Chiu, Stein, Imbra, Rahmsdorf, Jonat, Herrlich, and Karin, “Phorbol Ester-Inducible Genes Contain a Common cis Element Recognized by a TPA-Modulated Trans-acting Factor,” Cell, 49:729, 1987b.

Archibald, Krarup, Shefner, Li, Madison, “A collagen-based nerve guide conduit for peripheral nerve repair: An electrophysiological study of nerve regeneration in rodents and nonhuman primates,” J Comp Neurol, 306:685-696, 1991.

Atchison and Perry, “Tandem Kappa Immunoglobulin Promoters are Equally Active in the Presence of the Kappa Enhancer: Implications for Model of Enhancer Function,” Cell, 46:253, 1986.

Ausubel et al., (1996) Current Protocols in Molecular Biology. Current Protocols (Greene & Wiley). Harvard Medical School.

Baichwal and Sugden, “Vectors for gene transfer derived from animal DNA viruses: Transient and stable expression of transferred genes,” In: Kucherlapati R, ed. Gene transfer. New York: Plenum Press, pp. 117-148, 1986.

Banerji, Olson, and Schaffner, “A Lymphocyte-Specific Cellular Enhancer is Located Downstream of the Joining Region in Immunoglobulin Heavy-Chain Genes,” Cell, 35:729, 1983.

Berkhout, Silverman, and Jeang, “Tat Trans-activates the Human Immunodeficiency Virus Through a Nascent RNA Target,” Cell, 59:273, 1989.

Blanar, Baldwin, Flavell, and Sharp, “A Gamma-Interferon-Induced Factor That Binds the Interferon Response Sequence of the MHC Class I Gene, H-2Kb,” EMBO J., 8:1139, 1989.

Bodine and Ley, “An Enhancer Element Lies 3′ to the Human A Gamma Globin Gene,” EMBO J., 6:2997, 1987.

Boshart, Weber, Jahn, Dorsch-Hasler, Fleckenstein, and Schaffner, “A Very Strong Enhancer is Located Upstream of an Immediate Early Gene of Human Cytomegalovirus,” Cell, 41:521, 1985.

Bosze, Thiesen, and Charnay, “A Transcriptional Enhancer with Specificity for Erythroid Cells is Located in the Long Terminal Repeat of the Friend Murine Leukemia Virus,” EMBO J., 5:1615, 1986.

Braddock, Chambers, Wilson, Esnouf, Adams, Kingsman, and Kingsman, “HIV-I Tat Activates Presynthesized RNA In the Nucleus,” Cell, 58:269, 1989.

Bulla and Siddiqui, “The Hepatitis B Virus Enhancer Modulates Transcription of the Hepatitis B Virus Surface-Antigen Gene From an Internal Location,” J. Virol., 62:1437, 1986.

Caley et al., “Humoral, mucosal, and cellular immunity in response to a human immunodeficiency virus type 1 immunogen expressed by a Venezuelan equine encephalitis virus vaccine vector,” J. Virology, 71:3031-3038, 1997.

Caley et al., “Venezuelan equine encephalitis virus vectors expressing HIV-1 proteins: vector design strategies for improved vaccine efficacy,” Vaccine, 17:3124-3135, 1999.

Campbell and Villarreal, “Functional Analysis of the Individual Enhancer Core Sequences of Polyoma Virus: Cell-Specific Uncoupling of DNA Replication From Transcription,” Mol. Cell. Biol., 8:1993, 1988.

Campere and Tilghman, “Postnatal Repression of the .alpha.-fetoprotein Gene is Enhancer Independent,” Genes and Dev., 3:537, 1989.

Campo, Spandidos, Lang, and Wilkie, “Transcriptional Control Signals in the Genome of Bovine Papilloma Virus Type 1,” Nature, 303:77, 1983.

Carbonelli et al., “A plasmid vector for isolation of strong promoters in Escherichia coli,” Fems Microbiology Letters, 177:75-82, 1999.

Celander and Haseltine, “Glucocorticoid Regulation of Murine Leukemia Virus Transcription Elements is Specified by Determinants Within the Viral Enhancer Region,” J. Virology, 61:269, 1987.

Celander, Hsu, and Haseltine, “Regulatory Elements Within the Murine Leukemia Virus Enhancer Regions Mediate Glucocorticoid Responsiveness,” J. Virology, 62:1314, 1988.

Chandler, Maler, and Yamamoto, “DNA Sequences Bound Specifically by Glucocorticoid Receptor in vitro Render a Heterlogous Promoter Hormone Responsive in vivo,” Cell, 33:489, 1983.

Chang et al., “Foreign gene delivery and expression in hepatocytes using a hepatitis B virus vector,” Hepatology, 14:134A, 1991.

Chang, Erwin, and Lee, “Glucose-regulated Protein (GRP94 and GRP78) Genes Share Common Regulatory Domains and are Coordinately Regulated by Common Trans-acting Factors,” Mol. Cell. Biol., 9:2153, 1989.

Chatterjee, Lee, Rentoumis, and Jameson, “Negative Regulation of the Thyroid-Stimulating Hormone Alpha Gene by Thyroid Hormone: Receptor Interaction Adjacent to the TATA Box,” Proc Nat'l Acad Sci. USA, 86:9114, 1989.

Chen and Okayama, “High-efficiency transformation of mammalian cells by plasmid DNA,” Mol. Cell. Biol. 7:2745-2752, 1987.

Choi, Chen, Kriegler, and Roninson, “An Altered Pattern of Cross-Resistance in Multi-Drug-Resistant Human Cells Results From Spontaneous Mutations in the Mdr-1 (P-glycoprotein) Gene,” Cell, 53:519, 1988.

Clark, Voulgaropoulou, Fraley, and Johnson, “Cell lines for the production of recombinant adeno-associated virus,” Human Gene Therapy, 6:1329-1341, 1995.

Cocea, L. “Duplication of a region in the multiple cloning site of a plasmid vector to enhance cloning-mediated addition of restriction sites to a DNA fragment,” Biotechniques, 23:814-816, 1997.

Coffin, “Retroviridae and their replication,” In: Virology, Fields et al. (eds.), New York: Raven Press, pp. 1437-1500, 1990.

Cohen et al., “A repetitive sequence element 3′ of the human c-Ha-ras1 gene has enhancer activity”, J. Cell. Physiol., 5:75, 1987.

Costa, Lai, Grayson, and Darnell, “The Cell-Specific Enhancer of the Mouse Transthyretin (Prealbumin) Gene Binds a Common Factor at One Site and a Liver-Specific Factor(s) at Two Other Sites,” Mol. Cell. Biol., 8:81, 1988.

Couch et al., “Immunization with types 4 and 7 adenovirus by selective infection of the intestinal tract,” Am. Rev. Resp. Dis., 88:394-403, 1963.

Coupar et al., “A general method for the construction of recombinant vaccinia virus expressing multiple foreign genes,” Gene, 68:1-10, 1988.

Cripe, Haugen, Turk, Tabatabai, Schmid, Durst, Gissmann, Roman, and Turek, “Transcriptional Regulation of the Human Papilloma Virus-16 E6-E7 Promoter by a Keratinocyte-Dependent Enhancer, and by Viral E2 Trans-Activator and Repressor Gene Products: Implications for Cervical Carcinogenesis,” EMBO J., 6:3745, 1987.

Culotta and Hamer, “Fine Mapping of a Mouse Metallothionein Gene Metal-Response Element,” Mol. Cell. Biol., 9:1376, 1989.

Dandolo, Blangy, and Kamen, “Regulation of Polyma Virus Transcription in Murine Embryonal Carcinoma Cells,” J. Virology, 47:55, 1983.

Davis et al., “A viral vaccine vector that expresses foreign genes in lymph nodes and protects against mucosal challenge,” J. Virology, 70:3781-3787, 1996.

De Villiers, Schaffner, Tyndall, Lupton, and Kamen, “Polyoma Virus DNA Replication Requires an Enhancer,” Nature, 312:242, 1984.

Dellon and Mackinnon, “An alternative to the classical nerve graft for the management of the short nerve gap,” Plast Reconstr Surg, 82: 849-856, 1988.

den Dunner, Stokroos, Blaauw, Holwerda, Pennings, Robinson, Schakenraad, “Light-microscopic and electron-microscopic evaluation of short-term nerve regeneration using a biodegradable poly(DL-lactide- -caprolacton) nerve guide,” J Biomed Materials Research, 31:105-115,(1996).

Deschamps, Meijlink, and Verma, “Identification of a Transcriptional Enhancer Element Upstream From the Proto-Oncogene Fos,” Science, 230:1174, 1985.

Edbrooke, Burt, Cheshire, and Woo, “Identification of cis-Acting Sequences Responsible for Phorbol Ester Induction of Human Serum Amyloid A Gene Expression Via a Nuclear-Factor-.kappa.B-like Transcription Factor,” Mol. Cell. Biol., 9:1908, 1989.

Edlund, Walker, Barr, and Rutter, “Cell-Specific Expression of the Rat Insulin Gene: Evidence for Role of Two Distinct 5′ Flanking Elements,” Science, 230:912, 1985.

Evans, Brandt, Widmer, Gürlek, Savel, Gupta, Lohman, Williams, Hodges, Nabawi, Patrick, Mikos, “Tissue engineered conduits: The use of biodegradable poly(DL-lactic-co-gylcolic acid)(PLGA) scaffolds in peripheral nerve regeneration,” In: Biological Matrices and Tissue Reconstruction, Stark, Horch, Tanczos (eds). Berlin, Springer, 1998.

Evans, Brandt, Widmer, Lu, Meszlenyi, Gupta, Mikos, Hodges, Williams, Gürlek, Nabawi, Lohman, Patrick, “In vivo evaluation of poly(L-Lactic acid) porous conduits for peripheral nerve regeneration,” Biomaterials, 20:1109, 1999.

Fechheimer, Boylan, Parker, Sisken, Patel and Zimmer, “Transfection of mammalian cells with plasmid DNA by scrape loading and sonication loading,” Proc Nat'l. Acad. Sci. USA 84:8463-8467, 1987

Feng and Holland, “HIV-I Tat Trans-Activation Requires the Loop Sequence Within Tar,” Nature, 334:6178, 1988.

Firak and Subramanian, “Minimal Transcription Enhancer of Simian Virus 40 is a 74-Base-Pair Sequence that Has Interacting Domains,” Mol. Cell. Biol., 6:3667, 1986.

Flotte, Afione, Conrad, McGrath, Solow, Oka, Zeitlin, Guggino, and Carter, “Stable in vivo expression of the cystic fibrosis transmembrane conductance regulator with an adeno-associated virus vector,” Proc Nat'l Acad. Sci. USA, 90:10613-10617, 1993.

Flotte, Barraza-Ortiz, Solow, Afione, Carter, and Guggino, “An improved system for packaging recombinant adeno-associated virus vectors capable of in vivo transduction,” Gene Therapy, 2:29-37, 1995.

Foecking and Hofstetter, “Powerful and Versatile Enhancer-Promoter Unit for Mammalian Expression Vectors,” Gene, 45:101, 1986.

Fraley, Fomari and Kaplan, “Entrapment of a bacterial plasmid in phospholipid vesicles:potential for gene transfer,” Proc Nat'l. Acad. Sci. USA 76:3348-3352, 1979.

Friedmann, “Progress toward human gene therapy,” Science, 244:1275-1281, 1989.

Fujita, Shibuya, Hotta, Yamanishi, and Taniguchi, “Interferon-Beta Gene Regulation: Tandemly Repeated Sequences of a Synthetic 6-bp Oligomer Function as a Virus-Inducible Enhancer,” Cell, 49:357, 1987.

Ghosh and Bachhawat, “Targeting of liposomes to hepatocytes,” In. Wu G. and C. Wu ed. Liver diseases, targeted diagnosis and therapy using specific receptors and ligands. New York: Marcel Dekker, pp. 87-104, 1991.

Ghosh-Biswas et al., “Transgenic Indica rice (Oryza sativa L.) plants obtained by direct gene transfer to protoplasts,” J. Biotechnol., 32(1):1-10, 1994.

Ghosh-Choudhury et al., “Protein IX, a minor component of the human adenovirus capsid, is essential for the packaging of full-length genomes,” EMBO J., 6:1733-1739, 1987.

Gilles, Morris, Oi, and Tonegawa, “A Tissue-Specific Transcription Enhancer Element is Located in the Major Intron of a Rearranged Immunoglobulin Heavy-Chain Gene,” Cell, 33:717, 1983.

Gloss, Bernard, Seedorf, and Klock, “The Upstream Regulatory Region of the Human Papilloma Virus- 16 Contains an E2 Protein-Independent Enhancer Which is Specific for Cervical Carcinoma Cells and Regulated by Glucocorticoid Hormones,” EMBO J., 6:3735, 1987.

Gluck, “Ueber neuroplastik aut dem wege der transplantation,” Arch. Klin. Chir., 25:606-616, 1880.

Godbout, Ingram, and Tilghman, “Fine-Structure Mapping of the Three Mouse Alpha-Fetoprotein Gene Enhancers,” Mol. Cell. Biol., 8:1169, 1988.

Gomez-Foix et al., “Adenovirus-mediated transfer of the muscle glycogen phosphorylase gene into hepatocytes confers altered regulation of glycogen,” J. Biol. Chem., 267:25129-25134, 1992.

Goodbourn and Maniatis, “Overlapping Positive and Negative Regulatory Domains of the Human .beta.-Interferon Gene,” Proc. Nat'l Acad. Sci. USA, 85:1447, 1988.

Goodbourn, Burstein, and Maniatis, “The Human Beta-Interferon Gene Enhancer is Under Negative Control,” Cell, 45:601, 1986.

Gopal, “Gene transfer method for transient gene expression, stable transformation, and cotransformation of suspension cell cultures,” Mol. Cell. Biol. 5:1188-1190, 1985

Graham and Prevec, “Adenovirus-based expression vectors and recombinant vaccines,” Biotechnology, 20:363-390, 1992.

Graham and Prevec, “Manipulation of adenovirus vectors,” In. Gene Transfer and Expression Protocols, Murray, E. J., ed., Humana, New Jersey, vol. 7, 109-128, 1991.

Graham and Van Der Eb, “A new technique for the assay of infectivity of human adenovirus 5 DNA,” Virology 52:456-467, 1973.

Graham et al., “Characteristics of a human cell line transformed by DNA from human adenovirus type 5”, J. Gen. Virol., 36:59-72, 1977.

Greene, Bohnlein, and Ballard, “HIV-1, and Normal T-Cell Growth: Transcriptional Strategies and Surprises,” Immunology Today, 10:272, 1989.

Grosschedl and Baltimore, “Cell-Type Specificity of Immunoglobulin Gene Expression is Regulated by at Least Three DNA Sequence Elements,” Cell, 41:885, 1985.

Grunhaus and Horwitz, “Adenovirus as cloning vector,” Seminar in Virology, 3:237-252, 1992.

Harland and Weintraub, “Translation of mRNA injected into Xenopus oocytes is specifically inhibited by antisense RNA,” J. Cell Biol. 101:1094-1099, 1985.

Haslinger and Karin, “Upstream Promoter Element of the Human Metallothionein-II Gene Can Act Like an Enhancer Element,” Proc Nat'l Acad. Sci. USA, 82:8572, 1985.

Hauber and Cullen, “Mutational Analysis of the Trans-Activiation-Responsive Region of the Human Immunodeficiency Virus Type I Long Terminal Repeat,” J. Virology, 62:673, 1988.

Hen, Borrelli, Fromental, Sassone-Corsi, and Chambon, “A Mutated Polyoma Virus Enhancer Which is Active in Undifferentiated Embryonal Carcinoma Cells is not Repressed by Adenovirus-2 E1A Products,” Nature, 321:249, 1986.

Hensel, Meichle, Pfizenmaier, and Kronke, “PMA-Responsive 5′ Flanking Sequences of the Human TNF Gene,” Lymphokine Res., 8:347, 1989.

Hermonat and Muzyczka, “Use of adeno-associated virus as a mammalian DNA cloning vector; transduction of neomycin resistance into mammalian tissue culture cells,” Proc Nat'l Acad. Sci. USA, 81:6466-6470, 1984.

Herr and Clarke, “The SV40 Enhancer is Composed of Multiple Functional Elements That Can Compensate for One Another,” Cell, 45:461, 1986.

Hersdorffer et al., “Efficient gene transfer in live mice using a unique retroviral packaging line,” DNA Cell Biol., 9:713-723, 1990.

Herz and Gerard, “Adenovirus-mediated transfer of low density lipoprotein receptor gene acutely accelerates cholesterol clearance in normal mice,” Proc Nat'l Acad. Sci. USA 90:2812-2816, 1993.

Hirochika, Browker, and Chow, “Enhancers and Trans-Acting E2 Transcriptional Factors of Papilloma Viruses,” J. Virol., 61:2599, 1987.

Hirsch, Gaugler, Deagostini-Bauzin, Bally-Cuif, and Gordis, “Identification of Positive and Negative Regulatory Elements Governing Cell-Type-Specific Expression of the Neural-Cell-Adhesion-Molecule Gene,” Mol. Cell. Biol., 10:1959, 1990.

Holbrook, Gulino, and Ruscetti, “cis-Acting Transcriptional Regulatory Sequences in the Gibbon Ape Leukemia Virus (GALV) Long Terminal Repeat,” Virology, 157:211, 1987.

Horlick and Benfield, “The Upstream Muscle-Specific Enhancer of the Rat Muscle Creatine Kinase Gene is Composed of Multiple Elements,” Mol. Cell. Biol., 9:2396, 1989.

Horwich et al. “Synthesis of hepadenovirus particles that contain replication-defective duck hepatitis B virus genomes in cultured HuH7 cells,” J. Virol, 64:642-650, 1990.

Huang, Ostrowski, Berard, and Hagar, “Glucocorticoid Regulation of the Ha-MuSV p21 Gene Conferred by Sequences From Mouse Mammary Tumor Virus,” Cell, 27:245, 1981.

Hug, Costas, Staeheli, Aebi, and Weissmann, “Organization of the Murine Mx Gene and Characterization of its Interferon- and Virus-Inducible Promoter,” Mol. Cell. Biol., 8:3065, 1988.

Hwang, Lim, and Chae, “Characterization of the S-Phase-Specific Transcription Regulatory Elements in a DNA-Replication-Independent Testis-Specific H2B (TH2B) Histone Gene,” Mol. Cell. Biol., 10:585, 1990.

Imagawa, Chiu, and Karin, “Transcription Factor AP-2 Mediates Induction by Two Different Signal-Transduction Pathways: Protein Kinase C and cAMP,” Cell, 51:251, 1987.

Imbra and Karin, “Phorbol Ester Induces the Transcriptional Stimulatory Activity of the SV40 Enhancer,” Nature, 323:555, 1986.

Imler, Lemaire, Wasvlyk, and Waslyk, “Negative Regulation Contributes to Tissue Specificity of the Immunoglobulin Heavy-Chain Enhancer,” Mol. Cell. Biol, 7:2558, 1987.

Imperiale and Nevins, “Adenovirus 5 E2 Transcription Unit: an E1A-Inducible Promoter with an Essential Element that Functions Independently of Position or Orientation,” Mol. Cell. Biol., 4:875, 1984.

Jakobovits, Smith, Jakobovits, and Capon, “A Discrete Element 3′ of Human Immunodeficiency Virus 1 (HIV-1) and HIV-2 mRNA Initiation Sites Mediates Transcriptional Activation by an HIV Trans-Activator,” Mol. Cell. Biol., 8:2555, 1988.

Jameel and Siddiqui, “The Human Hepatitis B Virus Enhancer Requires Transacting Cellular Factor(s) for Activity,” Mol. Cell. Biol., 6:710, 1986.

Jaynes, Johnson, Buskin, Gartside, and Hauschka, “The Muscle Creatine Kinase Gene is Regulated by Multiple Upstream Elements, Including a Muscle-Specific Enhancer,” Mol. Cell. Biol., 8:62, 1988.

Johnson, Wold, and Hauschka, “Muscle Creatine Kinase Sequence Elements Regulating Skeletal and Cardiac Muscle Expression in Transgenic Mice,” Mol. Cell. Biol., 9:3393, 1989.

Jones and Shenk, “Isolation of deletion and substitution mutants of adenovirus type 5,” Cell, 13:181-188, 1978.

Kadesch and Berg, “Effects of the Position of the Simian Virus 40 Enhancer on Expression of Multiple Transcription Units in a Single Plasmid,” Mol. Cell. Biol., 6:2593, 1986.

Kaneda et al., “Increased expression of DNA cointroduced with nuclear protein in adult rat liver,” Science, 243:375-378, 1989.

Kaplitt, Leone, Samulski, Siao, Pfaff, O'Malley, and During, “Long-term gene expression and phenotypic correction suing adeno-associated virus vectors in the mammalian brain,” Nature Genetics, 8:148-154, 1994.

Karin, Haslinger, Heguy, Dietlin, and Cooke, “Metal-Responsive Elements Act as Positive Modulators of Human Metallothionein-IIA Enhancer Activity,” Mol. Cell. Biol., 7:606, 1987.

Karlsson et al., EMBO J., 5:2377-2385, 1986.

Katinka, Vasseur, Montreau, Yaniv, and Blangy, “Polyoma DNA Sequences Involved in the Control of Viral Gene Expression in Murine Embryonal Carcinoma Cells,” Nature, 290:720, 1981.

Katinka, Yaniv, Vasseur, and Blangy, “Expression of Polyoma Early Functions in Mouse Embryonal Carcinoma Cells Depends on Sequence Rearrangements in the Beginning of the Late Region,” Cell, 20:393, 1980.

Kato et al., “Expression of hepatitis .beta. virus surface antigen in adult rat liver,” J. Biol. Chem., 266:3361-3364, 1991.

Kawamoto, Makino, Niw, Sugiyama, Kimura, Anemura, Nakata, and Kakunaga, “Identification of the Human Beta-Actin Enhancer and its Binding Factor,” Mol. Cell. Biol., 8:267, 1988.

Kiledjian, Su, and Kadesch, “Identification and Characterization of Two Functional Domains Within the Murine Heavy-Chain Enhancer,” Mol. Cell. Biol., 8:145, 1988.

Klamut, Gangopadyhay, Worton, and Ray, “Molecular and Functional Analysis of the Muscle-Specific Promoter Region of the Duchenne Muscular Dystrophy Gene,” Mol. Cell. Biol., 10:193, 1990.

Klein et al., “High-velocity microprojectiles for delivering nucleic acids into living cells,” Nature, 327:70-73, 1987.

Koch, Benoist, and Mathis, “Anatomy of a New B-Cell-Specific Enhancer,” Mol. Cell. Biol., 9:303, 1989.

Kotin, Siniscalco, Samulski, Zhu, Hunter, McLaughlin, Muzyczka, and Bems, “Site-specific integration by adeno-associated virus,” Proc Natl Acad. Sci. USA, 87:2211-2215, 1990.

Kriegler and Botchan, In: Eukaryotic Viral Vectors, Y. Gluzman, ed., Cold Spring Harbor: Cold Spring Harbor Laboratory, NY, 1982.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In. Gene Expression, D. Hamer and M. Rosenberg, eds., New York: Alan R. Liss, 1983.

Kriegler, Perez, Defay, Albert and Liu, “A Novel Form of TNF/Cachectin Is a Cell-Surface Cytotoxix Transmembrane Protein: Ramifications for the Complex Physiology of TNF,” Cell, 53:45, 1988.

Kriegler, Perez, Hardy and Botchan, “Transformation Mediated by the SV40 T Antigens:

Separation of the Overlapping SV40 Early Genes with a Retroviral Vector,” Cell, 38:483, 1984a.

Kuhl, De La Fuenta, Chaturvedi, Parinool, Ryals, Meyer, and Weissman, “Reversible Silencing of Enhancers by Sequences Derived From the Human IFN-alpha Promoter,” Cell, 50:1057, 1987.

Kunz, Zimmerman, Heisig, and Heinrich, “Identification of the Promoter Sequences Involved in the Interleukin-6-Dependent Expression of the Rat Alpha-2-Macroglobulin Gene,” Nucl. Acids Res., 17:1121, 1989.

LaFace, Hermonat, Wakeland, and Peck, “Gene transfer into hematopoietic progenitor cells mediated by an adeno-associated virus vector,” Viology, 162:483-486, 1988.

Larsen, Harney, and Moore, “Repression Medaites Cell-Type-Specific Expression of the Rat Growth Hormone Gene,” Proc Nat'l Acad. Sci. USA., 83:8283, 1986.

Laspia, Rice, and Mathews, “HIV-1 Tat Protein Increases Transcriptional Initiation and Stabilizes Elongation,” Cell, 59:283, 1989.

Latimer, Berger, and Baumann, “Highly Conserved Upstream Regions of the α₁-Antitrypsin Gene in Two Mouse Species Govern Liver-Specific Expression by Different Mechanisms,” Mol. Cell. Biol., 10:760, 1990.

Laughlin, Cardellichio, and Coon, “Latent Infection of KB Cells with Adeno-Associated Virus Type 2,” J. Virol, 60:515-524, 1986.

Le Gal La Salle et al., “An adenovirus vector for gene transfer into neurons and glia in the brain,” Science, 259:988-990, 1993.

Lebkowski, McNally, Okarma, and Lerch, “Adeno-associated virus: a vector system for efficient introduction and integration of DNA into a variety of mammalian cell types,” Mol. Cell. Biol., 8:3988-3996, 1988.

Lee et al., “Glucocorticoids Regulate Expression of Dihydrofolate Reductase cDNA in Mouse Mammary Tumor Virus Chimaeric Plasmids,” Nature, 294:228, 1981.

Leitner et al., “Enhancement of tumor-specific immune response with plasmid DNA replicon Vectors,” Cancer Res.60:51-55, 2000.

Levenson et al., “Internal ribosomal entry site-containing retroviral vectors with green fluorescent protein and drug resistance markers,” Human Gene Therapy, 9:1233-1236, 1998.

Levinson et al., Nature, 295:79, 1982.

Levinson, Khoury, VanDeWoude, and Gruss, “Activation of SV40 Genome by 72-Base-Pair Tandem Repeats of Moloney Sarcoma Virus,” Nature, 295:79, 1982.

Levrero et al., “Defective and nondefective adenovirus vectors for expressing foreign genes in vitro and in vivo,” Gene, 101:195-202, 1991.

Lin, Cross, Halden, Dragos, Toledano, and Leonard, “Delineation of an Enhancerlike Positive Regulatory Element in the Interleukin-2 Receptor .alpha.-Chain Gene,” Mol. Cell. Biol., 10:850, 1990.

Lorz et al., Mol Gen Genet, 199:178-182, 1985.

Lundborg and Hansson, “Nerve regeneration through preformed pseudosynovial tubes,” J Hand Surg, 5(1):35-38, 1980.

Luo, Zhou, Cooper, Munshi, Boswell, Broxmeyer, and Srivastava, “Adeno-associated virus 2 mediated transfer and functional expression of a gene encoding the human granulocyte-macrophage colony-stimulating factor,” Blood,82 (Supp.): 1,303A, 1994.

Luria, Gross, Horowitz, and Givol, “Promoter Enhancer Elements in the Rearranged Alpha-Chain Gene of the Human T-Cell Receptor,” EMBO J., 6:3307, 1987.

Lusky and Botchan, “Transient Replication of Bovine Papilloma Virus Type 1 Plasmids: cis and trans Requirements,” Proc Nat'l Acad. Sci. USA, 83:3609, 1986.

Lusky and Botchan, Proc. Nat'l Acad. Sci. USA., 83:3609, 1986.

Lusky et al., Mol. Cell. Biol., 3:1108, 1983.

Lusky, Berg, Weiher, and Botchan, “Bovine Papilloma Virus Contains an Activator of Gene Expression at the Distal End of the Early Transcription Unit,” Mol. Cell. Biol. 3:1 108, 1983.

Macejak and Sarnow, “Internal initiation of translation mediated by the 5′ leader of a cellular mRNA,” Nature, 353:90-94, 1991.

Madison, da Silva, Dikkes, “Entubulation repair with protein additives increases the maximum nerve gap distance successfully bridged with tubular prostheses,” Brain Research, 447:325-334 (1988).

Majors and Varmus, Proc. Nat'l Acad. Sci. USA., 80:5866, 1983.

Mann et al., “Construction of a retrovirus packaging mutant and its use to produce helper-free defective retrovirus,” Cell, 33:153-159, 1983.

Markowitz et al., “A safe packaging line for gene transfer: Separating viral genes on two different plasmids,” J. Virol, 62:1120-1124, 1988.

McCarty, Christensen, and Muzyczka, “Sequences Required for Coordinate Induction of Adeno-Associated Virus p19 and p40 Promoters by Rep Protein,” J. Virol, 65:2936-2945, 1991.

McLaughlin, Collis, Hermonat, and Muzyczka, “Adeno-Associated Virus General Transduction Vectors: Analysis of Proviral Structures,” J. Virol., 62:1963-1973, 1988.

McNeall, Sanchez, Gray, Chesterman, and Sleigh, “Hyperinducible Gene Expression From a Metallotionein Promoter Containing Additional Metal-Responsive Elements,” Gene, 76:81, 1989.

Mikos, Throsen, Czerwonka, Bao, Langer, Winslow, Vacanti, “Preparation and characterization of poly(L-lactic acid) foams,” Polymer, 35:1068-1077, 1994.

Miksicek, Heber, Schmid, Danesch, Posseckert, Beato, and Schutz, “Glucocorticoid Responsiveness of the Transcriptional Enhancer of Moloney Murine Sarcoma Virus,” Cell, 46:203, 1986.

Mordacq and Linzer, “Co-localization of Elements Required for Phorbol Ester Stimulation and Glucocorticoid Repression of Proliferin Gene Expression,” Genes and Dev., 3:760, 1989.

Moreau, Hen, Wasylyk, Everett, Gaub, and Chambon, “The SV40 Base-Repair Repeat Has a Striking Effect on Gene Expression Both in SV40 and Other Chimeric Recombinants,” Nucl. Acids Res., 9:6047, 1981.

Musesing, Smith, and Capon, “Regulation of mRNA Accumulation by a Human Immunodeficiency Virus Trans-Activator Protein,” Cell, 48:691, 1987.

Muzyczka, “Use of Adeno-Associated Virus as a General Transduction Vector for Mammalian Cells,” Curr. Top. Microbiol. Immunol., 158:97-129, 1992.

Ng, Gunning, Liu, Leavitt, and Kedes, “Regulation of the Human Beta-Actin Promoter by Upstream and Intron Domains,” Nuc. Acids Res. 17:601, 1989.

Nicolas and Rubinstein, “Retroviral vectors,” In: Vectors: A survey of molecular cloning vectors and their uses, Rodriguez and Denhardt (eds.), Stoneham: Butterworth, pp. 494-513, 1988.

Nicolau and Sene, “Liposome-mediated DNA transfer in eukaryotic cells: dependence of the transfer efficiency upon the type of liposomes used and the host cell cycle stage,” Biochim. Biophys. Acta 721:185-190, 1982.

Nicolau et al., “Liposomes as carriers for in vivo gene transfer and expression,” Methods Enzymol., 149:157-176, 1987.

Ohi, Dixit, Tillery, and Plonk, “Construction and replication of an adeno-associated virus expression vector that contains human .lambda.-globin cDNA,” Gene, 89L:279-282, 1990.

Omirulleh et al., “Activity of a chimeric promoter with the doubled CaMV 35S enhancer element in protoplast-derived cells and transgenic plants in maize.” Plant Mol. Biol., 21:415-28, 1993.

Ondek, Sheppard, and Herr, “Discrete Elements Within the SV40 Enhancer Region Display Different Cell-Specific Enhancer Activities,” EMBO J., 6:1017, 1987.

Omitz, Hammer, Davison, Brinster, and Palmiter, “Promoter and Enhancer Elements From the Rat Elastase I Gene Function Independently of Each Other and of Heterologous Enhancers,” Mol. Cell. Biol. 7:3466, 1987.

Palmiter, Chen, and Brinster, “Differential Regulation of Metallothionein-Thymidine Kinase Fusion Genes in Transgenic Mice and Their Offspring,” Cell, 29:701, 1982.

Paskind et al., “Dependence of moloney murine leukemia virus production on cell growth,” Virology, 67:242-248, 1975.

Pech, Rao, Robbins, and Aaronson, “Functional Identification of Regulatory Elements Within the Promoter Region of Platelet-Derived Growth Factor 2,” Mol. Cell. Biol., 9:396, 1989.

Pelletier and Sonenberg, “Internal initiation of translation of eukaryotic mRNA directed by a sequence derived from poliovirus RNA,” Nature, 334:320-325, 1988.

Perez-Stable and Constantini, “Roles of Fetal .gamma.-globin Promoter Elements and the Adult β-globin 3′ Enhancer in the Stage-Specific Expression of Globin Genes,” Mol. Cell. Biol., 10:1116, 1990.

Picard and Schaffner, “A Lymphocyte-Specific Enhancer in the Mouse Immunoglobulin Kappa Gene,” Nature, 307:83, 1984.

Pinkert, Ornitz, Brinster, and Palmiter, “An Albumin Enhancer Located 10 kb Upstream Functions Along With its Promoter to Direct Efficient, Liver-Specific Expression in Transgenic Mice,” Genes and Dev., 1:268, 1987.

Ponta, Kennedy, Skroch, Hynes, and Groner, “Hormonal Response Region in the Mouse Mammary Tumor Virus Long Terminal Repeat Can Be Dissociated From the Proviral Promoter and Has Enhancer Properties,” Proc. Nat'l Acad. Sci. USA, 82:1020, 1985.

Porton, Zaller, Lieberson, and Eckhardt, “Immunoglobulin Heavy-Chain Enhancer is Required to Maintain Transfected .gamma.2A Gene Expression in a pre-B-cell Line,” Mol. Cell. Biol., 10:1076, 1990.

Potrykus et al., Mol. Gen. Genet., 199:183-188, 1985.

Potter et al., “Enhancer-dependent expression of human k immunoglobulin genes introduced into mouse pre-B lymphocytes by electroporation,” Proc Nat'l Acad. Sci. USA, 81:7161-7165, 1984.

Queen and Baltimore, “Immunoglobulin Gene Transcription is Activated by Downstream Sequence Elements,” Cell, 35:741, 1983.

Quinn, Farina, Gardner, Krutzsch, and Levens, “Multiple Components are Required for Sequence Recognition of the AP1 Site in the Gibbon Ape Leukemia Virus Enhancer,” Mol. Cell. Biol., 9:4713, 1989.

Racher et al., Biotechnology Techniques, 9:169-174, 1995.

Ragot et al., “Efficient adenovirus-mediated transfer of a human minidystrophin gene to skeletal muscle of mdx mice,” Nature, 361:647-650, 1993.

Redondo, Hata, Brocklehurst, and Krangel, “A T-Cell-Specific Transcriptional Enhancer Within the Human T-Cell Receptor .delta. Locus,” Science, 247:1225, 1990.

Reisman and Rotter, “Induced Expression From the Moloney Murine Leukemia Virus Long Terminal Repeat During Differentiation of Human Myeloid Cells is Mediated Through its Transcriptional Enhancer,” Mol. Cell. Biol., 9:3571, 1989.

Resendez Jr., Wooden, and Lee, “Identification of Highly Conserved Regulatory Domains and Protein-Binding Sites in the Promoters of the Rat and Human Genes Encoding the Stress-Inducible 78-kilodalton Glucose-Regulated Protein,” Mol. Cell. Biol., 8:4579, 1988.

Rich et al., “Development and analysis of recombinant adenoviruses for gene therapy of cystic fibrosis,” Hum. Gene Ther., 4:461-476, 1993.

Ridgeway, “Mammalian expression vectors,” In: Rodriguez R L, Denhardt D T, ed. Vectors: A survey of molecular cloning vectors and their uses. Stoneham: Butterworth, pp. 467-492, 1988.

Ripe, Lorenzen, Brenner, and Breindl, “Regulatory Elements in the 5′ Flanking Region and the First Intron Contribute to Transcriptional Control of the Mouse alpha-1-type Collagen Gene,” Mol. Cell Biol., 9:2224, 1989.

Rippe, Brenner and Leffert, “DNA-mediated gene transfer into adult rat hepatocytes in primary culture,” Mol. Cell Biol., 10:689-695, 1990.

Rittling, Coutinho, Amarm, and Kolbe, “AP-1/jun-binding Sites Mediate Serum Inducibility of the Human Vimentin Promoter,” Nuc. Acids Res., 17:1619, 1989.

Rosen, Sodroski, and Haseltine, “The Location of cis-acting Regulatory Sequences in the Human T-Cell Lymphotropic Virus Type III (HTLV-111/LAV) Long Terminal Repeat,” Cell, 41:813, 1988.

Rosenfeld, Siegfried, Yoshimura, Yoneyama, Fukayama, Stier, Paakko, Gilardi, Stratford-Perricaudet, Perricaudet, Jallat, Pavirani, Lecocq, Crystal, “Adenovirus-mediated transfer of a recombinant .alpha. 1-antitrypsin gene to the lung epithelium in vivo,” Science, 252:431-434, 1991.

Rosenfeld, Yoshimura, Trapnell, Yoneyama, Rosenthal, Dalemans, Fukayama, Bargon, Stier, Stratford-Perricaudet, Perricaudet, Guggino, Pavirani, Lecocq, Crystal, “In vivo transfer of the human cystic fibrosis transmembrane conductance regulator gene to the airway epithelium,” Cell, 68:143-155,1992.

Roux et al., “A versatile and potentially general approach to the targeting of specific cell types by retroviruses: Application to the infection of human cells by means of major histocompatibility complex class I and class II antigens by mouse ecotropic murine leukemia virus-derived viruses,” Proc Nat'l Acad. Sci. USA, 86:9079-9083, 1989.

Sakai, Helms, Carlstedt-Duke, Gustafsson, Rottman, and Yamamoto, “Hormone-Mediated Repression: A Negative Glucocorticoid-Response Element From the Bovine Prolactin Gene,” Genes and Dev., 2:1144, 1988.

Sambrook, J., Fritsch, E. F., and Maniatis, T. (1989). Molecular cloning: a laboratory manual. Cold Spring Harbor Laboratory Press, Cold Spring Harbor, New York.

Samulski, Chang, and Shenk, “Helper-free stocks of recombinant adeno-associated viruses: Normal integration does not require viral gene expression,” J. Virol, 63:3822-3828, 1989.

Samulski, Zhu, Xiao, Brook, Housman, Epstein, and Hunter, “Targeted integration of adeno-associated virus (AAV) into human chromosome 19,” EMBO J, 10:3941-3950, 1991.

Satake, Furukawa, and Ito, “Biological Activities of Oligonucleotides Spanning the F9 Point Mutation Within the Enhancer Region of Polyoma Virus DNA,” J. Virology, 62:970, 1988.

Schaffner, Schirm, Muller-Baden, Wever, and Schaffner, “Redundancy of Information in Enhancers as a Principle of Mammalian Transcription Control,” J. Mol. Biol., 201:81, 1988.

Searle, Stuart, and Palmiter, “Building a Metal-Responsive Promoter With Synthetic Regulatory Elements,” Mol. Cell. Biol., 5:1480, 1985.

Seckel et.al., “Nerve regeneration through synthetic biodegradable nerve guides: Regulation by the target organ,” Plast Reconstr Surg 74:173-181, 1984.

Sharp and Marciniak, “HIV Tar: an RNA Enhancer?,” Cell, 59:229, 1989.

Shaul and Ben-Levy, “Multiple Nuclear Proteins in Liver Cells are Bound to Hepatitis B Virus Enhancer Element and its Upstream Sequences,” EMBO J., 6:1913, 1987.

Shelling and Smith, “Targeted integration of transfected and infected adeno-associated virus vectors containing the neomycin resistance gene,” Gene Therapy, 1:165-169, 1994.

Sherman, Basta, Moore, Brown, and Ting, “Class II Box Consensus Sequences in the HLA-DR.alpha. Gene: Transcriptional Function and Interaction with Nuclear Proteins,” Mol. Cell. Biol.,9:50, 1989.

Sleigh and Lockett, “SV40 Enhancer Activation During Retinoic-Acid-Induced Differentiation of F9 Embryonal Carcinoma Cells,” J. EMBO, 4:3831, 1985.

Spalholz, Yang, and Howley, “Transactivation of a Bovine Papilloma Virus Transcriptional Regulatory Element by the E2 Gene Product,” Cell, 42:183, 1985.

Spandau and Lee, “Trans-Activation of Viral Enhancers by the Hepatitis B Virus X Protein,” J. Virology, 62:427, 1988.

Spandidos and Wilkie, “Host-Specificities of Papilloma Virus, Moloney Murine Sarcoma Virus and Simian Virus 40 Enhancer Sequences,” EMBO J., 2:1193, 1983.

Stephens and Hentschel, “The Bovine Papilloma Virus Genome and its Uses as a Eukaryotic Vector,” Biochem. J., 248:1, 1987.

Stratford-Perricaudet and Perricaudetp. 51-61, In: Human Gene Transfer, Eds, O. Cohen-Haguenauer and M. Boiron, Editions John Libbey Eurotext, France, 1990.

Stratford-Perricaudet et al., Hum. Gene. Ther. 1:241-256,1991.

Stuart, Searle, and Palmiter, “Identification of Multiple Metal Regulatory Elements in Mouse Metallothionein-I Promoter by Assaying Synthetic Sequences,” Nature, 317:828, 1985.

Sullivan and Peterlin, “Transcriptional Enhancers in the HLA-DQ Subregion,” Mol. Cell. Biol., 7:3315, 1987.

Swartzendruber and Lehman, “Neoplastic Differentiation: Interaction of Simian Virus 40 and Polyoma Virus with Murine Teratocarcinoma Cells,” J. Cell. Physiology, 85:179, 1975.

Takebe, Seiki, Fujisawa, Hoy, Yokota, Arai, Yoshida, and Arai, “SR.alpha. Promoter: An Efficient and Versatile Mammalian cDNA Expression System Composed of the Simian Virus 40 Early Promoter and the R-U5 Segment of Human T-Cell Leukemia Virus Type 1 Long Terminal Repeat,” Mol. Cell. Biol., 8:466, 1988.

Tavernier, Gheysen, Duerinck, Can Der Heyden, and Fiers, “Deletion Mapping of the Inducible Promoter of Human IFN-beta Gene,” Nature, 301:634, 1983.

Taylor, Solomon, Weiner, Paucha, Bradley, and Kingston, “Stimulation of the Human Heat-Shock Protein 70 Promoter in vitro by Simian Virus 40 Large T Antigen,” J. Biol. Chem., 264:15160, 1989.

Temin, “Retrovirus vectors for gene transfer: Efficient integration into and expression of exogenous DNA in vertebrate cell genome,” In: Gene Transfer, Kucherlapati (ed.), New York: Plenum Press, pp. 149-188, 1986.

Thiesen, Bosze, Henry, and Chamay, “A DNA Element Responsible for the Different Tissue Specificities of Friend and Moloney Retroviral Enhancers,” J. Virology, 62:614, 1988.

Thomas, D. “Nanotechnology's many disciplines,” Bio/Technology, 13(5):439-443, 1995.

Top et al., “Immunization with live types 7 and 4 adenovirus vaccines. II. Antibody response and protective effect against acute respiratory disease due to adenovirus type 7,” J. Infect. Dis., 124:155-160, 1971.

Tratschin, Miller, Smith, and Carter, “Adeno-associated virus vector for high-frequency integration, expression and rescue of genes in mammalian cells,” Mol. Cell. Biol., 5:32581-3260, 1985.

Tratschin, West, Sandbank, and Carter, “A human parvovirus, adeno-associated virus, as a eucaryotic vector: transient expression and encapsidation of the procaryotic gene for chloramphenicol acetyltransferase,” Mol. Cell. Biol., 4:2072-2081, 1984.

Treisman, “Transient Accumulation of c-fos RNA Following Serum Stimulation Requires a Conserved 5′ Element and c-fos 3′ Sequences,” Cell, 42:889, 1985.

Tronche, Rollier, Bach, Weiss, and Yaniv, “The Rat Albumin Promoter: Cooperation with Upstream Elements is Required When Binding of APF/HNF 1 to the Proximal Element is Partially Impaired by Mutation or Bacterial Methylation,” Mol. Cell. Biol., 9:4759, 1989.

Tronche, Rollier, Herbomel, Bach, Cereghini, Weiss, and Yaniv, “Anatomy of the Rat Albumin Promoter,” Mol. Biol. Med., 7:173, 1990.

Trudel and Constantini, “A 3′ Enhancer Contributes to the Stage-Specific Expression of the Human Beta-Globin Gene,” Genes and Dev., 6:954, 1987.

Tur-Kaspa, Teicher, Levine, Skoultchi and Shafritz, “Use of electroporation to introduce biologically active foreign genes into primary rat hepatocytes,” Mol. Cell Biol., 6:716-718, 1986.

Tyndall, La Mantia, Thacker, Favaloro, and Kamen, “A Region of the Polyoma Virus Genome Between the Replication Origin and Late Protein-Coding Sequences is Required in cis for Both Early Gene Expression and Viral DNA Replication,” Nuc. Acids. Res., 9:6231, 1981.

Valentini, Aebischer, Winn, Galletti, “Collagen-and laminin-containing gels impede peripheral nerve regeneration through semipermeable nerve guidance channels,” Experimental neurology, 98:350-356, 1987.

Vannice and Levinson, “Properties of the Human Hepatitis B Virus Enhancer: Position Effects and Cell-Type Nonspecificity,” J. Virology, 62:1305, 1988.

Vasseur, Kress, Montreau, and Blangy, “Isolation and Characterization of Polyoma Virus Mutants Able to Develop in Multipotential Murine Embryonal Carcinoma Cells,” Proc Nat'l Acad. Sci. USA, 77:1068, 1980.

Walsh, Nienhuis, Samulski, Brown, Miller, Young, and Liu, “Phenotypic correction of Fanconi anemia in human hematopoietic cells with a recombinant adeno-associated virus vector,” J. Clin. Invest, 94:1440-1448, 1994.

Wang and Calame, “SV40 Enhancer-Binding Factors are Required at the Establishment but not the Maintenance Step of Enhancer-Dependent Transcriptional Activation,” Cell, 47:241, 1986.

Wang, Costas, Jones, Miller, Seckel, “Sleeve insertion and collagen coating improve nerve regeneration through vein conduits,” J Reconstruc Microsurg, 9(1): 39-48, 1992.

Watanabe et al., “Gene transfection of mouse primordial germ cells in vitro and analysis of their survival and growth control, Experimental Cell Research, 230:76-83, 1997.

Weber, De Villiers, and Schaffner, “An SV40 ‘Enhancer Trap’ Incorporates Exogenous Enhancers or Generates Enhancers From its Own Sequences,” Cell, 36:983, 1984.

Wei, Wei, Samulski, and Barranger, “Expression of the human glucocerebrosidase and arylsulfatase A genes in murine and patient primary fibroblasts transduced by an adeno-associated virus vector,” Gene Therapy, 1:261-268, 1994.

Weinberger, Jat, and Sharp, “Localization of a Repressive Sequence Contributing to B-cell Specificity in the Immunoglobulin Heavy-Chain Enhancer,” Mol. Cell. Biol., 8:988, 1984.

Widmer, Gupta, Lu, Meszlenyi, Evans, Brandt, Savel, Gürlek, Patrick, Mikos, “Manufacture of porous biodegradable polymer conduits by an extrusion process for guided tissue regeneration,” Biomaterials, 19:1945, 1998.

Williams, Danielsen Muller, Varon, “Exogenous matrix precursors promote functional nerve regeneration across a 15-mm gap within a silicone chamber in the rat, J Comparative Neurology, 264:284-290, 1987.

Winoto and Baltimore, αβ-lineage-specific Expression of the .alpha. T-Cell Receptor Gene by Nearby Silencers,” Cell, 59:649, 1989.

Wong et al., “Appearance of .beta.-lactamase activity in animal cells upon liposome mediated gene transfer,” Gene, 10:87-94, 1980.

Yang, Burkholder, Roberts, Martinell and McCabe, “In vivo and in vitro gene transfer to mammalian somatic cells by particle bombardment,” Proc Nat'l Acad Sci. USA, 87:9568-9572, 1990.

Yang, Chen, Trempe, “Characterization of cell lines that inducibly express the adeno-associated virus Rep proteins,” J. Virol, 68:4847-4856, 1994.

Yoder, Kang, Zhou, Luo, and Srivastava, “In vivo gene transfer in murine hematopoietic reconstituting stem cells mediated by the adeno-associated virus 2-based vectors,” Blood, 82 (Supp.): 1:347A, 1994.

Yutzey, Kline, and Konieczny, “An Internal Regulatory Element Controls Troponin I Gene Expression,” Mol. Cell. Biol, 9:1397, 1989.

Zhou, Broxmyer, Cooper, Harrington, and Srivastava “Adeno-associated virus 2 mediated gene transfer in murine hematopoietic cells, Exp. Hematol (N.Y.), 21:928-933, 1993.

Zhou, Cooper, Kang, Ruggieri, Heimfeld, Srivastava, and Broxmeyer, “Adeno-associated virus 2-mediated high efficiency gene transfer into immature and mature subsets of hematopoietic progenitor cells in human umbilical cord blood,” J.Exp.Med., 179:1867-1875, 1994.

What is claimed is:

1. A method for regenerating nerve tissue in vivo comprising:
(a) providing a device comprising
(i) a biodegradable conduit comprising at least two openings and a passage connecting said openings,
(ii) helper cells transformed with an expression cassette comprising a promoter, active in said cells, that directs the expression of a polynucleotide encoding a growth factor, wherein said cells are disposed within said passage, and
(b) implanting said device in a subject such that each of said openings are adjacent to nerve tissues,
whereby said nerve tissues are stimulated to regenerate into said passage by said growth factor produced by said cells.

2. The method of claim 1, wherein said cells are fibroblast cells, stem cells, fat cells, Schwann cells, astrocytes, endothelial cells and ex vivo propagated nerve cells.

3. The method of claim 1, wherein said biodegradable conduit is comprised of PLGA or PLLA.

4. The method of claim 1, wherein the growth factor expression is inducible.

5. The method of claim 1, wherein said growth factor is Nerve Growth Factor (NGF), Fibroblast Growth Factor (FGF), Brain-Derived Neurotrophic Factor (BDNF), GDNF, VEGF, neurotrophin 3, or neurotrphin 4-5.

6. The method of claim 4, wherein inducible growth factor expression is driven by administration of Muristerone A, GS-E, or tetracycline.

7. The method of claim 6, wherein administration is intravenous, intrathecal, intracavitary and by catheter.

8. The method of claim 1, wherein said cells further comprise a cell kill gene that renders said cells susceptible to killing following administration of a substance.

9. The method of claim 8, wherein said cell kill gene is an enzyme and said substance is a prodrug.

10. The method of claim 9, wherein said cell kill gene comprises a promoter selected from the group consisting of CMV IE, SV40, HSV tk, RSV LTR, EF-1α and ubiquitin.

11. The method of claim 9, wherein said cell kill gene is thymidine kinase.

12. The method of claim 8, wherein said cell kill gene is a toxin and said substance is an activator of the transcription of said cell kill gene.

13. The method of claim 8, further comprising the step of administering said substance to said subject in an amount sufficient to kill said cells.

14. The method of claim 13, wherein administration is by is intravenous, intrathecal, intracavitary and by catheter.

15. The method of claim 1, wherein said promoter is selected from the group consisting of CMV IE, SV40, HSV tk, RSV LTR, EF-1α or ubiquitin.

16. The method of claim 1, wherein said expression construct further comprises a polyadenylation signal.

17. The method of claim 1, wherein said expression construct further comprises a selectable marker.

18. The method of claim 1, wherein said expression construct further comprises a screenable marker.

19. The method of claim 1, wherein said subject is a human.

20. The method of claim 6, wherein the induction is maintained for 24 hours.

21. The method of claim 6, wherein the induction is maintained for 48 hours.

22. The method of claim 6, wherein the induction is maintained for four days.

23. The method of claim 6, wherein the induction is maintained for seven days.

24. The method of claim 6, wherein the induction is maintained for ten days.

25. An implantable device comprising:
(a) a biodegradable conduit comprising at least two openings and a passage connecting said openings; and
(b) cells transformed with an expression cassette comprising a promoter, active in eukaryotic cells, that directs the expression of a polynucleotide encoding a growth factor, wherein said cells are disposed within said passage.

26. The device of claim 25, wherein said cells are fibroblast cells, stem cells, fat cells, Schwann cells, astrocytes, endothelial cells and ex vivo propagated nerve cells.

27. The device of claim 25, wherein said biodegradable conduit is comprised of PLGA or PLLA.

28. The device of claim 25, wherein the growth factor expression is inducible.

29. The device of claim 25, wherein the growth factor is Nerve Growth Factor (NGF), Fibroblast Growth Factor (FGF), Brain-Derived Neurotrophic Factor (BDNF), GDNF, VEGF, neurotrophin 3, or neurotrphin 4-5.

30. The device of claim 28, wherein inducible growth factor expression is driven by Muristerone A, GS-E, or tetracycline.

31. The device of claim 30, wherein administration is intravenous, intrathecal, intracavitary and by catheter.

32. The device of claim 25, wherein said cells further comprise a cell kill gene that renders said cells susceptible to killing following administration of a substance.

33. The device of claim 32, wherein said cell kill gene is an enzyme and said substance is a prodrug.

34. The device of claim 33, wherein said cell kill gene comprises a promoter selected from the group consisting of CMV IE, SV40, HSV tk, RSV LTR, EF-1α or ubiquitin.

35. The device of claim 33, wherein said cell kill gene is thymidine kinase.

36. The device of claim 32, wherein said cell kill gene is a toxin and said substance is an activator of the transcription of said cell kill gene.

37. The device of claim 25, wherein said expression construct further comprises a polyadenylation signal.

38. The device of claim 25, wherein said expression construct further comprises a selectable marker.

39. The device of claim 25, wherein said expression construct further comprises a screenable marker.

40. A kit comprising an implantable device comprising:
(a) a biodegradable conduit comprising at least two openings and a passage connecting said openings; and
(b) cells transformed with an expression cassette comprising a promoter, active in eukaryotic cells, that directs the expression of a polynucleotide encoding a growth factor, wherein said cells are disposed within said passage.

41. The kit of claim 40, wherein said promoter is inducible, and said kit further comprises an inducer of said promoter in a suitable container.

42. The kit of claim 40, wherein said cells further comprise a cell kill gene that renders said cells susceptible to killing following administration of a substance, and said kit further comprises said substance in a suitable container.

43. The kit of claim 40, wherein said cells are fibroblast cells, stem cells, fat cells, Schwann cells, astrocytes, endothelial cells and ex vivo propagated nerve cells.

44. The kit of claim 43, wherein said cells are fibroblast cells.

45. The kit of claim 44, wherein said fibroblast cells are dermal fibroblast cells.

46. The kit of claim 40, wherein said growth factor is Nerve Growth Factor (NGF), Fibroblast Growth Factor (FGF), Brain-Derived Neurotrophic Factor (BDNF), GDNF, VEGF, neurotrophin 3, or neurotrphin 4-5.
